(19) United States
(12) Patent Application Publication
(10) Pub. No.: US 2018/0362616 A1
(43) Pub. Date: Dec. 20, 2018

(54) VARIANTS OF AMYLOID beta-PROTEIN PRECURSOR INHIBITOR DOMAIN

(71) Applicant: THE NATIONAL INSTITUTE FOR BIOTECHNOLOGY IN THE NEGEV LTD., Beer-Sheva (IL)

(72) Inventors: Niv PAPO, Ra'anana (IL); Itay COHEN, Dimona (IL); Amiram SANANES, Hevel Lakhish (IL)

(21) Appl. No.: 16/060,361

(22) PCT Filed: Dec. 8, 2016
(86) PCT No.: PCT/IL2016/051318

§ 371 Date: Jun. 7, 2018

Related U.S. Application Data
(60) Provisional application No. 62/265,719, filed on Dec. 10, 2015.
(60) Provisional application No. 62/313,824, filed on Mar. 28, 2016.

(51) Int. Cl. C07K 14/81; A61K 38/55; A61P 35/04

(52) U.S. Cl. CPC C07K 14/8114; A61K 51/08; A61P 35/04; A61K 38/55

(57) ABSTRACT

Variants of amyloid β-protein precursor inhibitor domain (APPI), effective in inhibiting mesotrypsin and/or kallikrein-6, and composition comprising same, are provided. Further, methods of use of said peptides or composition, including, but not limited to treatment of cancer are provided.

### CROSS-REFERENCE TO RELATED APPLICATIONS

This application claims the benefit of priority of U.S. Provisional Patent Application No. 62/265,719, filed Dec. 10, 2015, and U.S. Provisional Patent Application No. 62/313,824, filed Mar. 28, 2016, the contents of which are incorporated herein by reference in their entirety.

### FIELD OF INVENTION

This invention is directed to; inter alia, peptides derived from amyloid β-protein precursor inhibitor domain (APPI), effective in inhibiting specific serine proteases such as mesotrypsin and/or kallikrein-6, and methods of use thereof, including, but not limited to treatment of malignant diseases.

### BACKGROUND OF THE INVENTION

Human amyloid β-protein precursor inhibitor (APPI), also known as protease nexin-2, is the secreted form of amyloid β-protein precursor (APP). APPI contains a Kunitz serine protease inhibitor domain known as KPI (Kunitz Protease Inhibitor).

Serine proteases are enzymes that cleave peptide bonds in proteins, in which serine serves as the nucleophilic amino acid at the enzyme's active site. Serine proteases are involved in a variety of metabolic pathways. In humans, they are responsible for coordinating various physiological functions, including digestion, immune response, blood coagulation and reproduction. Several studies have demonstrated that abnormal regulation of specific serine proteases play a role in pathological conditions such as genesis of malignant tumors and metastasis invasiveness. Human Kallikrein 6 (hK6) is a member of the human Kallikrein serine proteases family. It is a 223 amino acids protease with trypsin-like activity, having an Arginine-specific digestive capability. Studies have shown that hK6 high expression is highly correlated with genesis of many kinds of malignant tumors, including breast, colon and ovary. In these and other studies, hK6 was found to mediate proliferation, migration and invasiveness of the malignant cells. Moreover, it was shown to have a significant role in brain malignancies, by digestion of myelin. In cancer models inhibition of hK6 resulted in less aggressive behavior of cancer cells. In multiple sclerosis (MS) model hK6 inhibition resulted in delayed onset and reduced severity of symptoms. Thus, there is a need for highly specific inhibitors for hK6 that inhibit hK6 proteolytic activity, while avoiding its self-cleavage by hK6.

Among the human serine proteases currently known to be involved in pathological conditions, mesotrypsin is unusual and distinctly challenging in terms of elucidating mechanism of action and designing efficacious inhibitors. Although its specific pathological roles are yet to be fully elucidated, the dysregulation and overexpression of mesotrypsin correlate with poor prognosis in many human tumors and with malignant behaviors in cancer models, making this protein an attractive target for therapeutic intervention. Mesotrypsin is particularly attractive as a target in metastatic prostate cancer, where in patients it is upregulated in metastatic tumors and associated with recurrence and metastasis, while in cell culture and orthotopic mouse models, it is found to drive invasive and metastatic phenotypes (Hockla, A., et al., Mol Cancer Res, 2012. 10(12): p. 1555-66). Likewise, in pancreatic cancer, mesotrypsin expression correlates with poorer patient survival, and in cell culture and animal models is found to promote cancer cell proliferation, invasion, and metastasis (Jiang, G., et al., Gut, 2010. 59(11): p. 1535-44). Potent and selective inhibitors of mesotrypsin could offer promise for treatment of patients with aggressive metastatic cancers, and would also offer tools to better dissect mesotrypsin function in cancer progression and metastasis.

During the last decade, mesotrypsin has emerged as a significant player in different stages of cancer development, and has been associated with cell malignancy in multiple cancers including lung, colon, breast, pancreas and prostate cancers. Early studies of transendothelial migration in non-small cell lung cancer (NSCLC) cultures showed mesotrypsin overexpression to be associated with invasion and metastasis, while comparative microarray assays of cells taken from NSCLC patients showed mesotrypsin overexpression to be predictive of poor survival.

Developing inhibitors that would target mesotrypsin presents special challenges, especially as this enzyme is resistant to inhibition by many polypeptide serine protease inhibitors, and further cleaves and inactivates many such inhibitors as physiological substrates. An additional challenge is presented by the need for selective inhibitors, since mesotrypsin shows high sequence homology and structural similarity with the major digestive trypsins (cationic and anionic trypsin), as well as with other serine proteases including kallikreins and coagulation factors. It is thus not surprising that there are currently no effective inhibitory agents with high stability, affinity and specificity to human mesotrypsin.

Although mesotrypsin and other trypsins share the same residues that contribute to their specificity, mesotrypsin exhibits unique sequence and structural features that contribute to its distinct resistance towards trypsin inhibitors. This resistance is most notably the result of two evolutionary mutations in mesotrypsin: the substitution of Gly-193 by Arg, which clashes sterically with the inhibitors, and the substitution of Tyr-39 by Ser, which prevents the formation of a hydrogen bond within the mesotrypsin/inhibitor complexes. These mutations are thus responsible for the unusually low affinity of mesotrypsin (relative to typical trypsins) for polypeptide trypsin inhibitors. They are also responsible for the more surprising ability of mesotrypsin to cleave several canonical trypsin inhibitors at an accelerated rate. This unique feature of mesotrypsin can be explained by the fact that, in contrast to other typical trypsins, the inhibitor affinity for mesotrypsin—and not its cleavage—is the rate-limiting step. The weakening of favorable interactions (Tyr39Ser) and the promotion of unfavorable interactions (Gly193Arg) between mesotrypsin and the canonical binding loop of the inhibitor results in expulsion of the binding loop from the active site upon cleavage of the inhibitor from mesotrypsin, thereby hindering re-association of the cleaved inhibitor.

The most striking example of the dramatic differences in proteolytic stability and binding affinity of serine protease inhibitors toward mesotrypsin is that of the differences between the human amyloid precursor protein inhibitor domain (APPI) and bovine pancreatic trypsin inhibitor (BPTI), both of which are natural Kunitz serine protease inhibitors. Although both serve as potential inhibitors of mesotrypsin, BPTI is the more stable of the two (Knecht, W., et al., J Biol Chem, 2007. 282(36): p. 26089-100). APPI is cleaved very rapidly, with a kinetic profile more closely resembling that of a substrate (Radisky, E. S., et al., Biochemistry, 2003. 42(21): p. 6484-92). The two inhibitors also display striking differences in mesotrypsin affinity, with APPI being 100-fold more tightly bound to the protease than BPTI (Grishina, Z., et al., Br J Pharmacol, 2005. 146(7): p. 990-9). There exists a long-felt need for more effective means of treating or ameliorating malignant diseases.

### SUMMARY OF THE INVENTION

The present invention provides amyloid precursor protein inhibitor domain (APPI) variants, and pharmaceutical compositions comprising same. The invention further provides methods of treating, ameliorating or inhibiting mesotrypsin- and/or Kallikrein-6-associated malignancies, including but not limited to prostate cancer.

According to one aspect, the present invention provides an isolated polypeptide comprising the amino acid sequence of SEQ ID NO: 1:

(EVCSEQAEXIGPCRAX₂X₃X₄RWYFDVTEGX₅CAPFX₆YGGCGGNRNNFDTEEYCMAVCG SAI) wherein:

X₁ is threonine, serine, cysteine or valine; X₂ is glycine, cysteine, leucine, histidine, serine, phenylalanine or alanine; X₃ is phenylalanine, leucine, tyrosine or tryptophan; X₄ is serine or phenylalanine; X₅ is lysine, isoleucine, leucine or methionine; and X₆ is valine, cysteine, isoleucine, leucine or methionine, or a fragment, a derivative or analog thereof.

According to another embodiment, the isolated polypeptide comprises the amino acid of SEQ ID NO: 2, wherein X₁ is threonine, serine, cysteine or valine; X₂ is glycine, cysteine or alanine; X₃ is phenylalanine, leucine, tyrosine or tryptophan; X₄ is serine; X₅ is lysine, isoleucine, leucine or methionine; and X₆ is valine, cysteine, isoleucine, leucine or methionine, or a fragment, a derivative or analog thereof.

According to another embodiment, the isolated polypeptide comprises the amino acid of SEQ ID NO: 3, wherein: X₁ is threonine or valine; X₂ is glycine; X₃ is phenylalanine; X₄ is serine; X₅ is lysine or leucine; and X₆ is valine, or a fragment, a derivative or analog thereof.

According to another embodiment, the isolated polypeptide molecule comprises the amino acid of SEQ ID NO: 4, wherein X₁ is cysteine, valine or threonine; X₂ is glycine or cysteine; X₃ is phenylalanine; X₄ is serine; X₅ is lysine or leucine; and X₆ is cysteine, or a fragment, a derivative or analog thereof.

According to another embodiment, the isolated polypeptide molecule comprises the amino acid of SEQ ID NO: 5, wherein X₁ is threonine; X₂ is glycine, leucine, histidine, serine or phenylalanine; X₃ is phenylalanine; X₄ is serine or phenylalanine; X₅ is lysine; and X₆ is valine, or a fragment, a derivative or analog thereof.

According to another embodiment, the isolated polypeptide molecule comprises the amino acid of SEQ ID NO: 6, wherein X₁ is threonine; X₂ is glycine or leucine; X₃ is phenylalanine; X₄ is serine or phenylalanine; X₅ is lysine; and X₆ is valine, or a fragment, a derivative or analog thereof.

According to another embodiment, the isolated polypeptide molecule comprises the amino acid of SEQ ID NO: 7, wherein X₁ is threonine; X₂ is leucine; X₃ is phenylalanine; X₄ is serine or phenylalanine; X₅ is lysine; and X₆ is valine, or a fragment, a derivative or analog thereof.

According to another embodiment, the isolated polypeptide comprises the amino acid of SEQ ID NO: 8, or a fragment, a derivative or analog thereof.

According to another embodiment, the isolated polypeptide comprises the amino acid of SEQ ID NO: 9, or a fragment, a derivative or analog thereof.

According to another embodiment, the isolated polypeptide comprises the amino acid of SEQ ID NO: 10, or a fragment, a derivative or analog thereof.

According to another embodiment, the isolated polypeptide comprises the amino acid of SEQ ID NO: 11, or a fragment, a derivative or analog thereof.

According to another embodiment, the isolated polypeptide comprises the amino acid of SEQ ID NO: 12, or a fragment, a derivative or analog thereof.

According to another embodiment, the isolated polypeptide comprises the amino acid of SEQ ID NO: 13, or a fragment, a derivative or analog thereof.

According to another embodiment, the isolated polypeptide comprises the amino acid of SEQ ID NO: 14, or a fragment, a derivative or analog thereof.

According to another embodiment, the polypeptide has a length of at most 80 amino acid residues. According to another embodiment, said analog has at least 95% sequence identity to SEQ ID NO: 1. According to another embodiment, said analog differs by at least one amino acid residue compared to SEQ ID NO: 25.

According to another aspect, there is provided a pharmaceutical composition comprising the polypeptide of the invention and a pharmaceutically acceptable carrier.

According to another aspect, there is provided a method for treating cancer in a subject in need thereof, the method comprising the step of administering to said subject a pharmaceutical composition comprising an effective amount of an isolated polypeptide comprising the amino acid selected from the group consisting of SEQ ID NO: 1-23, and a pharmaceutically acceptable carrier, thereby treating cancer in a subject in need thereof.

According to another aspect, the invention provides a pharmaceutical composition comprising an effective amount of an isolated polypeptide comprising the amino acid selected from the group consisting of SEQ ID NO: 1-14, and a pharmaceutically acceptable carrier, for use in treating cancer in a subject in need thereof.

According to another aspect, the invention provides use of a pharmaceutical composition comprising an effective amount of an isolated polypeptide comprising the amino acid selected from the group consisting of SEQ ID NO: 1-14 and a pharmaceutically acceptable carrier, for preparation of a medicament for treating cancer in a subject in need thereof.

According to another embodiment, the pharmaceutical composition comprises an effective amount of an isolated polypeptide comprising the amino acid selected from the group consisting of SEQ ID NO: 1-14, and a pharmaceutically acceptable carrier.

According to another embodiment, said cancer is a mesotrypsin-associated cancer. According to another embodiment, said cancer is selected from the group consisting of prostate, lung, colon, breast, pancreas and non-small cell lung cancer (NSCLC) or metastasis thereof. According to another embodiment, said cancer is prostate cancer.

According to another embodiment, said treating is inhibiting invasiveness of a cancerous cell.

According to another aspect, there is provided a method for imaging a mesotrypsin associated and/or kallikrein associated neoplastic tissue in a subject in need thereof, the method comprising the steps of:
administering an imaging reagent compound comprising: an effective amount of an amino acid molecule comprising the amino acid selected from the group consisting of SEQ ID NOs: 1-14, and an imaging agent to a subject, wherein said imaging reagent compound distributes in vivo; and
detecting the compound in said subject,
thereby imaging mesotrypsin associated and/or kallikrein-6 associated neoplastic tissue.

According to another aspect, there is provided kit comprising a composition comprising an amino acid molecule comprising the amino acid sequence selected from the group consisting of SEQ ID NOs: 1-14 or an analog, a derivative or fragment thereof. In some embodiments, the kit further comprises at least one signal producing label.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-D. APPI_{WT} is expressed, cleaved and detected by active and inactive mesotrypsin variants in yeast surface display (YSD) system. (A) Dual-color flow cytometric expression and folding analysis. APPI expression is shown on the X axis and binding of APPI to bovine-trypsin (50 nM) on the Y axis. Subpanels (1-4) represent unstained, (PE)-labeled expression, (FITC)-labeled binding and dual-labeled cells (demonstrating expression and binding, respectively). (B) APPI_{WT} is cleaved by mesotrypsin with a high off rate. The figure shows dual-labeled cells as in panel A, but with different concentrations of (FITC)-labeled active mesotrypsin. (C) General scheme of the “Triple staining method” for the detection of uncleaved APPI. (D) Intact APPI is detected by inactive mesotrypsin. The figure shows dual-labeled cells as in panel B but with the addition of inactive mesotrypsin (active and inactive mesotrypsin marked in red and blue, respectively). Here, the concentration of intact APPI correlated with the concentration of active mesotrypsin added to the sample. For all panels, the surface expression of APPI was detected using a primary antibody against the C-terminal c-Myc tag and a (PE)-labeled secondary antibody, while binding to APPI was detected by a biotinylated target (bovine trypsin or mesotrypsin) and (FITC)-labeled streptavidin. Non-induced cells are located in the bottom left quadrant of each plot.

FIGS. 2A-C. Identification of APPI clones with improved resistance to cleavage. (A) Stability maturation of the APPI library. The figure shows a flow activated cell sorting (FACS) of single or dual-labeled cells for expression (S0 and S1) or both expression and binding (S1 to S5), respectively. Here, the expressed population of APPI variants was sorted (S0), and the expression of the library was tested after enrichment (S1). Next, each cycle of stability maturation (S2 to S5) was performed with elevated concentrations of active mesotrypsin (as noted in in the upper right quadrant of each plot) and fixed concentration of inactive mesotrypsin (2 μM). Sorting gates are marked in red. (B) ‘Triple staining’ (B₁) and ‘double staining’ (B₂) analysis of APPI maturation cycles. The y-axis represents mean fluorescence intensity normalization of binding to expression. Data was analyzed using KaleidaGraph software with a sigmoidal curve fit. (C) ‘Double staining’ analysis of M17G, I18F, and F34V variants together with their combinations. A leftward shift in the sigmoid shape indicates a higher affinity whereas higher values of binding in the saturation of each variants indicates a higher proteolytic stability. The y-axis represents mean fluorescence intensity normalization of binding to expression. Data was analyzed using KaleidaGraph software, with a sigmoidal curve fit. For all panels, the surface expression of APPI was detected using a primary antibody against the C-terminal c-Myc tag and a (PE)-labeled secondary antibody, while binding to APPI was detected by biotinylated mesotrypsin and (FITC)-labeled streptavidin.

FIGS. 3A-H. Kinetics of mesotrypsin inhibition by APPI and hydrolysis of APPI by mesotrypsin. (A), Competitive patterns of mesotrypsin inhibition by APPI-M17G. Mesotrypsin cleavage of peptide substrate Z-GPR-pNA is competitively inhibited by APPI-M17G. (B), The Lineweaver-Burk double reciprocal transform of the data used in panel A. APPI (inhibitor) concentration is given at the top of each plot; mesotrypsin concentration was 0.25 nM. Data was fitted globally to the competitive inhibition equation using Prism, GraphPad Software. (C and E), Slow, tight binding inhibition of mesotrypsin by APPI. Steady-state equilibrium for the reactions of APPI-M17G and APPI-M17G/I18F/F34V with various concentrations of APPI and 145 μM of peptide substrate Z-GPR-pNA. (D and F), A re-plot of data from the binding curves shown in panels C and E, respectively, where V₀ is the uninhibited rate and Vᵢ is the rate in the presence of APPI, which allows calculation of Kᵢ using eq. 2 (as described in “Materials and Methods“under” Trypsin inhibition studies”). (G), Kinetics of APPI-M17G/I18F/F34V hydrolysis by mesotrypsin. Representative HPLC chromatograms are shown from a time course of APPI hydrolysis by mesotrypsin. Green and red peaks represent intact APPI and cleaved APPI, respectively. (H) Initial rate of hydrolysis, from which k_{cat} is calculated. Disappearance of intact APPI was quantified by integration of the HPLC peak in a time course that is illustrated in panel G. Hydrolysis reaction contained 50 μM of APPI and 2.5 μM of enzyme.

FIGS. 4A-B. “Triple mutant cycle analysis cube” that summarizing the additivity of free energy changes attributable to residue numbers 17, 18 and 34 on the APPI sequence. Each corner of the cube represents a different APPI variant, as annotated. (A), values along each edge represent ΔΔGₐ (kcal/mol), calculated using Equation 4; whereas each face of the cube represents a ΔΔGᵢₙₜ^{a} (kcal/mol) of a double mutant cycle attributable to the corner variants, calculated using Equation 3. Here, the equilibrium association constant that used is approximated as the reciprocal of the measured inhibition constant Ki. (B) figure shows the free energy changes as in panel A, but for catalysis (i.e. ΔΔG_{cat} and ΔΔGᵢₙₜ^{cat} respectively).

FIGS. 5A-B. Enhanced potency of APPI_{M17G/I18F/F34V} for inhibition of prostate cancer cell invasion. In Matrigel transwell invasion assays, shRNA knockdown of PRSS3 (KD) or treatment with inhibitors APPI_{WT} or APPI_{M17G/I18F/F34V} led to reductions in PC3-M cellular invasion compared to control cells. (A) images are shown for representative fields from stained invasion filters for (left to right) control cells, cells with PRSS3 knockdown (KD), cells treated with 10 nM APPI_{WT}, and cells treated with 10 nM APPI_{M17G/I18F/F34V}. (B) bar graph shows mean and S.E.M. for quadruplicate biological replicates. Black bars represent control cell samples, green bar represents cells with PRSS3 knockdown (KD), red bars represent cells treated with 10 nM inhibitor (APPI_{WT} or APPI_{M17G/I18F/F34V} as indicated), blue bars represent cells treated with 1 μM inhibitor (APPI_{WT} or APPI_{M17G/I18F/F34V} as indicated). **P<0.005 for t-test comparisons of indicated conditions versus control; *P=0.02 for t-test comparison of 10 nM treated conditions for APPI_{WT} vs. APPI_{M17G/I18F/F34V}.

FIG. 6. Protein validation. A representative example of the non-reduced SDS-PAGE of APPI_{WT} samples from gel-filtration (GF) with overlap on the GF chromatogram and the inhibitory effect on bovine trypsin catalytic activity. For the catalytic activity assay the inhibitor samples were diluted 1:1000 (inhibitory effect has no units i.e., normalized to the highest peak value).

FIGS. 7A-B. Representative nickel-IMAC purification of APPI_{WT}. The supernatant was loaded on a HisTrap (GE Healthcare) column for 24 h (Flowthrough; FT) using ÄKTApure instrument (GE Healthcare), followed by washing and elution (A). Gel filtration chromatography of APPI_{WT}. Eluted protein (2.5 ml) from the previous purification step was injected into a Superdex 75 16/600 column (GE Healthcare). The inset shows the elution time (ml) of the middle peak of different protein standards, including aprotinin (6.5 kDa), ribonuclease A (13.7 kDa), and ovalbumin (43 kDa). The Mw of the purified APPI was estimated to be 9.2 kDa according to the standards (B).

FIG. 8. Circular dichroism spectra. Absorbance was recorded over a range of 190-260 nm using a quartz cuvette with a path length of 1 mm. Three scans of 50 μM protein solutions were averaged to obtain smooth data and background corrected with respect to protein-free buffer. The inset presents a representative example of APPI-WT CD scans at room temperature (20° C.) and under denaturation (95° C.), and renaturation (at 20° C. following 95° C. incubation) conditions.

FIG. 9. Thermostability of APPI variants. Each variant (125 nM) was heated at 95° C. for 5 min and tested for its ability to inhibit the catalytic activity of bovine-trypsin (final concentrations of inhibitors and enzyme were 3.1 nM and 2.5 nM, respectively). Y axis represents the ratio of the % inhibitory effect of APPI after heating at 95° C. normalized by the % inhibitory effect of APPI before heating at 95° C.

FIG. 10. Evaluation of the clones in YSD. K_{D} differences between APPI WT (SEQ ID NO: 25), APPI 3M (SEQ ID NO: 8), APPI 3M G17L (SEQ ID NO: 14) and APPI 3M G17L,S19F (SEQ ID NO: 12) were determined and a titration curve was built. Binding was normalized to APPI expression on yeast cells.

FIG. 11. Determination of binding site. Both new clones (SEQ ID NOs: 13 and 14) were evaluated for their ability to bind hK6 at the presence of a small molecule which target the Ser residue within the active pocket of hK6. Binding was normalized to APPI expression on yeast cells.

FIG. 12. Evaluation of the clones in their soluble form. APPI WT (SEQ ID NO: 25) and APPI 3M (SEQ ID NO: 8) inhibited hK6 activity in low nano-Molar range having Ki=2.24 nM and Ki=1.1 nM, respectively. Each experiment was performed in triplicates.

FIG. 13. SPR results. APPIs were immobilized to a SPR nickel chip via the proteins His tag, and hK6 protein served as the analyte. The experiment was conducted at 25° C. APPI concentrations were 0.6125 nM, 1.25 nM, 2.5 nM, 5 nM, and 10 nM.

FIG. 14. APPI variant has no effect on AGS, HCT-116 nor SW-480 cell proliferation. Values are expressed as a duplicate average absorbance at 450 nm-690 nm.

FIGS. 15A-C. The APPI variant (SEQ ID NO: 13) inhibits cell invasion in AGS gastric cell line. Representative fields of invasive cells on membrane in the presence of a vehicle (A) or in the presence of 10 μM APPI (B), and a bar representing an average invasive cell number from 10 random fields in a triplicate (C), are shown.

### DETAILED DESCRIPTION OF THE INVENTION

The present invention provides amyloid precursor protein inhibitor domain (APPI) variants, and pharmaceutical compositions comprising same. The invention further provides methods of treating, ameliorating or inhibiting mesotrypsin-associated pathological conditions (e.g., malignancies, including but not limited to prostate cancer and/or Kallikrein-6-associated pathological conditions.

In some embodiments, the invention provides a method of reducing/inhibiting mesotrypsin activity and/or kallikrein-6 activity, the method comprises the step of contacting mesotrypsin and/or kallikrein-6 with the APPI variants of the invention. In some embodiments, the contacting is in vitro. In some embodiments, the contacting is in vivo.

According to some embodiments, the invention provides an APPI variant comprising at least one amino acid substitution compared to SEQ ID NO: 25 (EVCSEQAETGPCRAMISRWYFDVTEGKCAPFFYGGCGGNRNNFDTEEYCMAVCGSAI).

In some embodiments, the APPI variant comprises at least two amino acid substitutions compared to SEQ ID NO: 25. In some embodiments, the APPI variant comprises at least three amino acid substitutions compared to SEQ ID NO: 25. According to some embodiments, the APPI variant of the invention is selected from the amino acid sequences listed in Table 1 herein below.

TABLE 1
APPI variants of the invention

| Peptide | Amino acid sequence |
|---|---|
| SEQ ID NO: 1 | EVCSEQAEX₁GPCRAX₂X₃X₄RWYFDVTEGX₅CAPFX₆YGGCGGNRNNFDT |
| | EEYCMAVCGSAI |
| | X₁ = T, S, C, V; X₂ = G, C, A, L, H, S, F; X₃ = F, L, Y, W; X₄ = S, F; |
| | X₅ = K, I, L, M; X₆ = V, C, I, L, M; |
| SEQ ID NO: 2 | EVCSEQAEX₁GPCRAX₂X₃X₄RFDVTEGX₅CAPFX₆YGGCGGNRNNFFDT |
| | EEYCMAVCGSAI |
| | X₁ = T, S, C, V; X₂ = G, C, A; X₃ = F, L, Y, W; X₄ = S; X₅ = K, I, L, M; |
| | X₆ = V, C, I, L, M; |
| SEQ ID NO: 3 | EVCSEQAEX₁GPCRAX₂X₃X₄RWYFDVTEGX₅CAPFX₆YGGCGGNRNNFDT |
| | EEYCMAVCGSAI |
| | X₁ = T, V; X₂ = G; X₃ = F; X₄ = K, L; X₅ = V; |
| SEQ ID NO: 4 | EVCSEQAEX₁GPCRAX₂X₃X₄RWYFDVTEGX₅CAPFX₆YGGCGGNRNNFDT |
| | EEYCMAVCGSAI |
| | X₁ = C, T, V; X₂ = G, C; X₃ = F; X₄ = S; X₅ = K, L; X₆ = C; |
| SEQ ID NO: 5 | EVCSEQAEX₁GPCRAX₂X₃X₄RWYFDVTEGX₅CAPFX₆YGGCGGNRNNFDT |
| | EEYCMAVCGSAI |
| | X₁ = T; X₂ = G, L, H, S, F; X₃ = F; X₄ = S, F; X₅ = K; X₆ = V; |
| SEQ ID NO: 6 | EVCSEQAEX₁GPCRAX₂X₃X₄RWYFDVTEGX₅CAPFX₆YGGCGGNRNNFDT |
| | EEYCMAVCGSAI |
| | X₁ = T; X₂ = G, L; X₃ = F; X₄ = S, F; X₅ = K; X₆ = V; |
| SEQ ID NO: 7 | EVCSEQAEX₁GPCRAX₂X₃X₄RWYFDVTEGX₅CAPFX₆YGGCGGNRNNFDT |
| | EEYCMAVCGSAI |
| | X₁ = T; X₂ = L; X₃ = F; X₄ = S, F; X₅ = K; X₆ = V; |
| SEQ ID NO: 8 | EVCSEQAETGPCRAGFSRWYFDVTEGKCAPFVYGGCGGNRNNFDTEEYC |
| M17G_I18F_F34V | MAVCGSAI |
| SEQ ID NO: 9 | EVCSEQAENGPCRAGFSRWYFDVTEGKCAPFVYGGCGGNRNNFDTEEYC |
| T11V_M17G_ | MAVCGSAI |
| I118F_F34V | |
| SEQ ID NO: 10 | EVCSEQAETGPCRAGFSRWYFDVTEGLCAPFVYGGCGGNRNNFDTEEYC |
| M17G_I18F_ | MAVCGSAI |
| K29L_F34V | |
| SEQ ID NO: 11 | EVCSEQAECGPCRAGFSRWYFDVTEGKCAPFCYGGCGGNRNNFDTEEYC |
| T11C_M17G_ | MAVCGSAI |
| I18F_F34C | |
| SEQ ID NO: 12 | EVCSEQAETGPCRACFSRWYFDVTEGKCAPFCYGGCGGNRNNFDTEEYC |
| M17C_I18F_F34C | MAVCGSAI |
| SEQ ID NO: 13 | EVCSEQAETGPCRALFFRWYEDVTEGKCAPFVYGGCGGNRNNFDTEEYC |
| M17L, I118F, S1917, | MAVCGSAI |
| F34V | |
| SEQ ID NO: 14 | EVCSEQAETGPCRALFSRWYFDVTEGKCAPFVYGGCGGNRNNFDTEEYC |
| M17L, II8F, F34V | MAVCGSAI |
| SEQ ID NO: 15 | EVCSEQAETGPCRAHFSRWYFDVTEGKCAPFVYGGCGGNRNNFDTEEYC |
| M17H, I18F, F34V | MAVCGSAI |
| SEQ ID NO: 16 | EVCSEQAETGPCRASFSRWYFDVTEGKCAPFVYGGCGGNRNNFDTEEYC |
| M17S, I18F, F34V | MAVCGSAI |
| SEQ ID NO: 17 | EVCSEQAETGPCRAFFSRWYFDVTEGKCAPFVYGGCGGNRNNFDTEEYC |
| M17F, I18F, F34V | MAVCGSAI |
| SEQ ID NO: 18 | EVCSEQAETGPCRAGISRWYFDVTEGKCAPFFYGGCGGNRNNFDTEEYC |
| M17G | MAVCGSAI |
| SEQ ID NO: 19 | EVCSEQAETGPCRAMFSRWYEDVTEGKCAPFFYGGCGGNRNNFDTEEYC |
| I18F | MAVCGSAI |
| SEQ ID NO: 20 | EATSEQAETGPCRAMFSRWYFDVTEGKCAPFVYGGCGGNRNNFDTEEYC |
| I18F_F34V | MAVCGSAI |
| SEQ ID NO: 21 | EVCSEQAETGPCRAGISRWYTDVTEGKCAPFVYGGCGGNRNNFDTEEYC |
| M17G_F34V | MAVCGSAI |
| SEQ ID NO: 22 | EVCSEQAETGPCRAGFSRWYEDVTEGKCAPFFYGGCGGNRNNFDTEEYC |
| M17G_I18F | MAVCGSAI |
| SEQ ID NO: 23 | EVCSEQAETGPCRAMISRWYFDVTEGKCAPFVYGGCGGNRNNFDTEEYC |
| F34V | MAVCGSAI |

The present invention is based, in part, on the surprising finding that the APPI variants disclosed herein specifically bind mesotrypsin with substantially greater affinity than WT APPI and reduces or inhibits activity thereof. As exemplified in the example section below, the APPI variants disclosed herein exhibit higher stability than WT APPI. As further exemplified in the example section below, the APPI variants disclosed herein exhibit enhanced potency for inhibition of mesotrypsin-dependent cancer cells invasiveness. The present invention is further based, in part, on the surprising finding that some of the APPI variants disclosed herein further bind kallikrein-6 with substantially greater affinity than WT APPI and reduces or inhibits activity of both mesotrypsin and kallikrein-6 with substantially greater affinity than WT APPI. As exemplified in the example section below, these APPI variants significantly lower invasiveness of gastric cancer cells.

According to another embodiment, the isolated polypeptide has a higher selectivity and/or binding affinity to mesotrypsin than WT APPI. According to another embodiment, the isolated polypeptide has a higher selectivity and/or binding affinity to kallikrein-6 than WT APPI. According to another embodiment, the isolated polypeptide has a higher selectivity and/or binding affinity to mesotrypsin and kallikrein-6 than WT APPI. According to another embodiment, the isolated polypeptide has a higher stability than WT APPI. According to another embodiment, the isolated polypeptide comprises higher specificity to mesotrypsin than to other trypsins.

In some embodiments, the APPI variants of the invention reduce or inhibit the activity of mesotrypsin. In some embodiments, the activity of mesotrypsin is reduced by at least 10%, 20%, 30%, 40%, 50%, 60%, 70%, 90%, 95%, or 100%. Each possibility represents a separate embodiment of the present invention. In some embodiments, the APPI variants reduce or inhibit mesotrypsin-dependent cancer cells invasiveness. In such embodiments, the invasiveness of mesotrypsin-dependent cancer cells is reduced by at least 10%, 20%, 30%, 40%, 50%, 60%, 70%, 90%, 95%, or 100%. Each possibility represents a separate embodiment of the present invention.

In some embodiments, the APPI variants of the invention further reduce or inhibit the activity of kallikrein-6. In some embodiments, the activity of kallikrein-6 is reduced by at least 10%, 20%, 30%, 40%, 50%, 60%, 70%, 90%, 95%, or 100%. Each possibility represents a separate embodiment of the present invention. In some embodiments, the APPI variants capable of reducing or inhibiting activity of mesotrypsin and kallikrein-6 reduce, ameliorate or inhibit mesotrypsin-associated diseases and/or kallikrein-6-associated disease. In some embodiments, the APPI variants capable of reducing or inhibiting activity of mesotrypsin and kallikrein-6 reduce, ameliorate or inhibit cancer cells invasiveness. In some embodiments, the APPI variants capable of reducing or inhibiting activity of mesotrypsin and kallikrein-6 reduce, ameliorate or inhibit mesotrypsin-dependent and/or kallikrein-6 dependent cancer cells invasiveness.

As used herein, such as in connection with selective binding affinity, “higher” and “substantially greater” are used interchangeably to refer to at least a two-fold, at least a three-fold, at least a four-fold or at least a five-fold increase in the selectivity to mesotrypsin than WT APPI.

The term “peptide” as used herein encompasses native peptides (degradation products, synthetic peptides or recombinant peptides), peptidomimetics (typically including non-peptide bonds or other synthetic modifications) and the peptide analogues peptoids and semipeptoids, and may have, for example, modifications rendering the peptides more stable while in the body or more capable of penetrating into cells.

The terms “polypeptide”, “amino acid molecule” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

The term “isolated” peptide refers to a peptide that is essentially free from contaminating cellular components, such as carbohydrate, lipid, or other proteinaceous impurities associated with the peptide in nature. Typically, a preparation of isolated peptide contains the peptide in a highly-purified form, i.e., at least about 80% pure, at least about 90% pure, at least about 95% pure, greater than 95% pure, or greater than 99% pure. Each possibility represents a separate embodiment of the present invention.

The present invention further provides fragments, analogs and chemical modifications of the APPI variants of the present invention as long as they are capable of binding mesotrypsin and/or modulating (e.g. reducing or inhibiting) mesotrypsin activity. In some embodiments, the fragments, analogs and chemical modifications of the APPI variants encompassed by the present invention are further capable of binding kallikrein-6 and/or modulating (e.g. reducing or inhibiting) kallikrein-6 activity.

The peptides may comprise additional amino acids, either at the peptide's N-terminus, at the peptide's C-terminus or both. In another embodiment, the peptide has a length of at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 amino acids. Each possibility represents a separate embodiment of the present invention. In another embodiment, the peptide has a length of at most 80 amino acids.

According to another embodiment, the APPI variants of the invention encompass truncated forms and/or fragments of any one of SEQ ID NOs: 1-14 as long as they are capable of binding mesotrypsin and/or modulating (e.g. reducing or inhibiting) mesotrypsin activity and/or binding kallikrein-6 and/or modulating kallikrein-6 activity. In some embodiments, the APPI variants comprises amino acids 9-32 of any one of SEQ ID NOs: 1-14 or an analog thereof. In another embodiment, the fragments or the truncated forms of APPI variants of the invention comprise at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 amino acids derived from any one of SEQ ID NOs: 1-14. Each possibility represents a separate embodiment of the present invention. In another embodiment, the fragments or the truncated forms of APPI variants of the invention comprise 20 to 57, 20 to 56, 20 to 55, 20 to 54, 20 to 53, 20 to 52, 20 to 51, 20 to 50, 20 to 49, 20 to 48, 20 to 47, 20 to 46, 20 to 45, 20 to 44, 20 to 43, 20 to 42, 20 to 41, 20 to 40, 20 to 39, 20 to 38, 20 to 37, 20 to 36, 20 to 35, 20 to 34, 20 to 33, 20 to 32, 24 to 57, 24 to 56, 24 to 55, 24 to 54, 24 to 53, 24 to 52, 24 to 51, 24 to 50, 24 to 49, 24 to 48, 24 to 47, 24 to 46, 24 to 45, 24 to 44, 24 to 43, 24 to 42, 24 to 41, 24 to 40, 24 to 39, 24 to 38, 24 to 37, 24 to 36, 24 to 35, 24 to 34, 24 to 33, 24 to 32, 26 to 57, 26 to 56, 26 to 55, 26 to 54, 26 to 53, 26 to 52, 26 to 51, 26 to 50, 26 to 49, 26 to 48, 26 to 47, 26 to 46, 26 to 45, 26 to 44, 26 to 43, 26 to 42, 26 to 41, 26 to 40, 26 to 39, 26 to 38, 26 to 37, 26 to 36, 26 to 35, 26 to 34, 26 to 33, 26 to 32, amino acids derived from any one of SEQ ID NOs: 1-14. Each possibility represents a separate embodiment of the present invention.

Conservative substitution of amino acids as known to those skilled in the art are within the scope of the present invention. Conservative amino acid substitutions include replacement of one amino acid with another having the same type of functional group or side chain e.g. aliphatic, aromatic, positively charged, negatively charged. One of skill will recognize that individual substitutions, deletions or additions to peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art.

The following six groups each contain amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W) (see, e.g., Creighton, Proteins, 1984).

The term “analog” includes any peptide having an amino acid sequence substantially identical to one of the sequences specifically shown herein in which one or more residues have been conservatively substituted with a functionally similar residue and which displays the abilities as described herein. Examples of conservative substitutions include the substitution of one non-polar (hydrophobic) residue such as isoleucine, valine, leucine or methionine for another, the substitution of one polar (hydrophilic) residue for another such as between arginine and lysine, between glutamine and asparagine, between glycine and serine, the substitution of one basic residue such as lysine, arginine or histidine for another, or the substitution of one acidic residue, such as aspartic acid or glutamic acid for another. Each possibility represents a separate embodiment of the present invention.

The phrase “conservative substitution” also includes the use of a chemically derivatized residue in place of a non-derivatized residue provided that such peptide displays the requisite function of modulating the immune system's innate response as specified herein.

The term “derived from” or “corresponding to” refers to construction of an amino acid sequence based on the knowledge of a sequence using any one of the suitable means known to one skilled in the art, e.g. chemical synthesis in accordance with standard protocols in the art.

According to another embodiment, the APPI variant of the invention has at least 75%, 80%, 85%, 90%, 91%, 92%, 93%, 94%, 95%, 96%, 97%, 98%, 99% sequence identity to any one of SEQ ID NO: 1-14. Each possibility represents a separate embodiment of the present invention. According to another embodiment, the APPI variant has at least 75% sequence identity to any one of SEQ ID NO: 1-14. According to another embodiment, the APPI variant has at least 80% sequence identity to any one of SEQ ID NO: 1-14. According to another embodiment, said APPI variant has at least 85% sequence identity to any one of SEQ ID NO: 1-14. According to another embodiment, said APPI variant has at least 90% sequence identity to any one of SEQ ID NO: 13. According to another embodiment, said APPI variant has at least 95% sequence identity to any one-of SEQ ID NO: 1-14. In some embodiments, the APPI variant of the invention comprises a sequence having at least 75%, 80%, 85%, 90%, 91%, 92%, 93%, 94%, 95%, 96%, 97%, 98%, or 99% identity to any one of SEQ ID NOs: 1-14, wherein the APPI variant: (i) binds mesotrypsin with substantially greater affinity than WT APPI and reduces activity thereof, and (ii) is capable of reducing, inhibiting or ameliorating a mesotrypsin associated pathological conditions (e.g., cancer). Each possibility represents a separate embodiment of the present invention. In some embodiments, the APPI variant further binds kallikrein-6 with substantially greater affinity than WT APPI and reduces activity thereof. In some embodiments, the APPI variant is capable of reducing, inhibiting or ameliorating a mesotrypsin associated and or kallikrein-6-associated pathological conditions.

As used herein, the term “APPI variant” includes at least one amino acid substitution with respect to the WT APPI (SEQ ID NO: 25). In some embodiments, the APPI variant includes at least two amino acid substitutions with respect to the WT APPI (SEQ ID NO: 25). In some embodiments, the APPI variant includes at least three amino acid substitutions with respect to the WT APPI (SEQ ID NO: 25). In some embodiments, the APPI variants of the invention include an amino acid substitution of methionine at position 15 of SEQ ID NO: 25 and at least one additional amino acid substitution. In some embodiments, the at least one additional amino acid substitution is a substitution of the amino acid at a position selected from: 9, 16, 17, 27 and 32 of SEQ ID NO: 25. In some embodiments, the APPI variants of the invention have the amino acid sequence of SEQ ID NO: 1 wherein at least one of X₁, X₃, X₄, X₅, and X₆ differs from the corresponding amino acid of SEQ ID NO: 25.

In some embodiments, the APPI variants of the invention have at least 50 folds, 60 folds, 70 folds, 80 folds, 90 folds, 100 folds, 150 folds, 200 folds, 250 folds, 300 folds, 400 folds, 500 folds, 600 folds, 700 folds, 800 folds, 900 folds, or 1000 folds decrease in Ki value for inhibiting mesotrypsin, relative to WT APPI. In some embodiments, the APPI variants of the invention have at least 50 folds, 60 folds, 70 folds, 80 folds, 90 folds, 100 folds, 150 folds, 200 folds, 250 folds, 300 folds, 400 folds, 500 folds, 600 folds, 700 folds, 800 folds, 900 folds, or 1000 folds decrease in Ki value for inhibiting kallikrein-6, relative to WT APPI.

As used herein “Ki” refers to an inhibition constant which represents the concentration required to produce half maximum inhibition of a target protein (e.g., enzyme such as mesotrypsin, kallikrein-6). The inhibition constant (Ki) is ordinarily used as a measure of capacity to inhibit enzyme activity, with a low Ki indicating a more potent inhibitor.

Percentage sequence identity can be determined, for example, by the Fitch et al. version of the algorithm (Fitch et al, Proc. Natl. Acad. Sci. U.S.A. 80: 1382-1386 (1983)) described by Needleman et al, (Needleman et al, J. Mol. Biol. 48: 443-453 (1970)), after aligning the sequences to provide for maximum homology. Alternatively, the determination of percent identity between two sequences can be accomplished using the mathematical algorithm of Karlin and Altschul (1993) Proc. Natl. Acad. Sci. USA 90:5873-5877. Such an algorithm is incorporated into the BLASTP program of Altschul et al. (1990) J. Mol. Biol. 215, 403-410. To obtain gapped alignments for comparative purposes, Gapped BLAST is utilized as described in Altschul et al (1997) Nucleic Acids Res. 25:3389-3402. When utilizing BLAST and Gapped BLAST programs, the default parameters of the respective programs (e.g., XBLAST) are used.

Typically, the present invention encompasses derivatives of the APPI peptides. The term “derivative” or “chemical derivative” includes any chemical derivative of the peptide having one or more residues chemically derivatized by reaction of side chains or functional groups. Such derivatized molecules include, for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as chemical derivatives are those peptides, which contain one or more naturally occurring amino acid derivatives of the twenty standard amino acid residues. For example: 4-hydroxyproline may be substituted for proline; 5-hydroxylysine may be substituted for lysine; 3-methylhistidine may be substituted for histidine; homoserine may be substituted or serine; and ornithine may be substituted for lysine.

In addition, a peptide derivative can differ from the natural sequence of the peptides of the invention by chemical modifications including, but are not limited to, terminal-NH2 acylation, acetylation, or thioglycolic acid amidation, and by terminal-carboxlyamidation, e.g., with ammonia, methylamine, and the like. Peptides can be either linear, cyclic or branched and the like, which conformations can be achieved using methods well known in the art.

The peptide derivatives and analogs according to the principles of the present invention can also include side chain bond modifications, including but not limited to —CH2-NH—, —CH2-S—, —CH2-S=0, OC—NH—, —CH2-O—, —CH2-CH2-, S═C—NH—, and —CH═CH—, and backbone modifications such as modified peptide bonds. Peptide bonds (—CO—NH—) within the peptide can be substituted, for example, by N-methylated bonds (—N(CH3)-CO—); ester bonds (—C(R)H—C-0-0-C(R)H—N); ketomethylene bonds (—CO—CH2-); a-aza bonds (—NH—N(R)—CO—), wherein R is any alkyl group, e.g., methyl; carba bonds (—CH2-NH—); hydroxyethylene bonds (—CH(OH)—CH2-); thioamide bonds (—CS—NH); olefmic double bonds (—CH═CH—); and peptide derivatives (—N(R)—CH2-CO—), wherein R is the “normal” side chain, naturally presented on the carbon atom. These modifications can occur at one or more of the bonds along the peptide chain and even at several (e.g., 2-3) at the same time.

The present invention also encompasses peptide derivatives and analogs in which free amino groups have been derivatized to form amine hydrochlorides, p-toluene sulfonylamino groups, carbobenzoxyamino groups, t-butyloxycarbonylamino groups, chloroacetylamino groups or formylamino groups. Free carboxyl groups may be derivatized to form, for example, salts, methyl and ethyl esters or other types of esters or hydrazides. The imidazole nitrogen of histidine can be derivatized to form N-im-benzylhistidine.

The peptide analogs can also contain non-natural amino acids. Examples of non-natural amino acids include, but are not limited to, sarcosine (Sar), norleucine, ornithine, citrulline, diaminobutyric acid, homoserine, isopropyl Lys, 3-(2′-naphtyl)-Ala, nicotinyl Lys, amino isobutyric acid, and 3-(3′-pyridyl-Ala).

Furthermore, the peptide analogs can contain other derivatized amino acid residues including, but not limited to, methylated amino acids, N-benzylated amino acids, O-benzylated amino acids, N-acetylated amino acids, O-acetylated amino acids, carbobenzoxy-substituted amino acids and the like. Specific examples include, but are not limited to, methyl-Ala (Me Ala), MeTyr, MeArg, MeGlu, MeVal, MeHis, N-acetyl-Lys, O-acetyl-Lys, carbobenzoxy-Lys, Tyr-O-Benzyl, Glu-O-Benzyl, Benzyl-His, Arg-Tosyl, t-butylglycine, t-butylalanine, phenylglycine, cyclohexylalanine, and the like.

The invention further includes peptide analogs, which can contain one or more D-isomer forms of the amino acids. Production of retro-inverso D-amino acid peptides where at least one amino acid, and perhaps all amino acids are D-amino acids is well known in the art. When all of the amino acids in the peptide are D-amino acids, and the N- and C-terminals of the molecule are reversed, the result is a molecule having the same structural groups being at the same positions as in the L-amino acid form of the molecule. However, the molecule is more stable to proteolytic degradation and is therefore useful in many of the applications recited herein. Diastereomeric peptides may be highly advantageous over all L- or all D-amino acid peptides having the same amino acid sequence because of their higher water solubility, lower immunogenicity, and lower susceptibility to proteolytic degradation. The term “diastereomeric peptide” as used herein refers to a peptide comprising both L-amino acid residues and D-amino acid residues. The number and position of D-amino acid residues in a diastereomeric peptide of the preset invention may be variable so long as the peptide is capable of displaying the requisite function binding and/or modulating (e.g. reducing or inhibiting) mesotrypsin activity, as specified herein.

The peptides of the invention may be synthesized or prepared by techniques well known in the art. The peptides can be synthesized by a solid phase peptide synthesis method of Merrifield (see J. Am. Chem. Soc, 85:2149, 1964). Alternatively, the peptides of the present invention can be synthesized using standard solution methods well known in the art (see, for example, Bodanszky, M., Principles of Peptide Synthesis, Springer-Verlag, 1984) or by any other method known in the art for peptide synthesis.

In general, these methods comprise sequential addition of one or more amino acids or suitably protected amino acids to a growing peptide chain bound to a suitable resin.

Normally, either the amino or carboxyl group of the first amino acid is protected by a suitable protecting group. The protected or derivatized amino acid can then be either attached to an inert solid support (resin) or utilized in solution by adding the next amino acid in the sequence having the complimentary (amino or carboxyl) group suitably protected, under conditions conductive for forming the amide linkage. The protecting group is then removed from this newly added amino acid residue and the next amino acid (suitably protected) is added, and so forth. After all the desired amino acids have been linked in the proper sequence, any remaining protecting groups are removed sequentially or concurrently, and the peptide chain, if synthesized by the solid phase method, is cleaved from the solid support to afford the final peptide.

In the solid phase peptide synthesis method, the alpha-amino group of the amino acid is protected by an acid or base sensitive group. Such protecting groups should have the properties of being stable to the conditions of peptide linkage formation, while being readily removable without destruction of the growing peptide chain. Suitable protecting groups are t-butyloxycarbonyl (BOC), benzyloxycarbonyl (Cbz), biphenylisopropyloxycarbonyl, t-amyloxycarbonyl, isobornyloxycarbonyl, (alpha,alpha)-dimethyl-3,5 dimethoxybenzyloxycarbonyl, o-nitrophenylsulfenyl, 2-cyano-t-butyloxycarbonyl, 9-fluorenylmethyloxycarbonyl (FMOC) and the like.

In the solid phase peptide synthesis method, the C-terminal amino acid is attached to a suitable solid support. Suitable solid supports useful for the above synthesis are those materials, which are inert to the reagents and reaction conditions of the stepwise condensation-deprotection reactions, as well as being insoluble in the solvent media used. Suitable solid supports are chloromethylpolystyrene-divinylbenzene polymer, hydroxymethyl-polystyrene-divinylbenzene polymer, and the like. The coupling reaction is accomplished in a solvent such as ethanol, acetonitrile, N,N-dimethylformamide (DMF), and the like. The coupling of successive protected amino acids can be carried out in an automatic polypeptide synthesizer as is well known in the art.

The peptides of the invention may alternatively be synthesized such that one or more of the bonds, which link the amino acid residues of the peptides are non-peptide bonds. These alternative non-peptide bonds include, but are not limited to, imino, ester, hydrazide, semicarbazide, and azo bonds, which can be formed by reactions well known to skilled in the art.

In some embodiments, recombinant protein techniques are used to generate the protein of the invention. In some embodiments, recombinant protein techniques are used for generation of relatively long peptides (e.g., longer than 18-25 amino acid). In some embodiments, recombinant protein techniques are used for the generation of large amounts of the protein of the invention. In some embodiments, recombinant techniques are described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680 and Brogli et al, (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463.

The peptides of the present invention, analogs or derivatives thereof produced by recombinant techniques can be purified so that the peptides will be substantially pure when administered to a subject. The term “substantially pure” refers to a compound, e.g., a peptide, which has been separated from components, which naturally accompany it.

Typically, a peptide is substantially pure when at least 50%, preferably at least 75%, more preferably at least 90%, and most preferably at least 99% of the total material (by volume, by wet or dry weight, or by mole percent or mole fraction) in a sample is the peptide of interest. Purity can be measured by any appropriate method, e.g., in the case of peptides by HPLC analysis.

According to another aspect, the present invention provides an isolated polynucleotide sequence encoding the polypeptides of the present invention, or an analog or a conjugate thereof.

The term “polynucleotide” means a polymer of deoxyribonucleic acid (DNA), ribonucleic acid (RNA) or a combination thereof, which can be derived from any source, can be single- or double-stranded, and can optionally contain synthetic, non-natural, or altered nucleotides, which are capable of being incorporated into DNA or RNA polymers.

An “isolated polynucleotide” refers to a polynucleotide segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to polynucleotides, which have been substantially purified from other components, which naturally accompany the polynucleotide in the cell, e.g., RNA or DNA or proteins. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA, which is part of a hybrid gene encoding additional polypeptide sequence, and RNA such as mRNA.

The term “encoding” refers to the inherent property of specific sequences of nucleotides in an isolated polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a peptide or protein if transcription and translation of mRNA corresponding to that gene produces the peptide or protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the peptide or protein or other product of that gene or cDNA.

One who is skilled in the art will appreciate that more than one polynucleotide may encode any given peptide or protein in view of the degeneracy of the genetic code and the allowance of exceptions to classical base pairing in the third position of the codon, as given by the so-called “Wobble rules.” It is intended that the present invention encompass polynucleotides that encode the peptides of the present invention as well as any analog thereof.

A polynucleotide of the present invention can be expressed as a secreted peptide where the polypeptide of the present invention or analog thereof is isolated from the medium in which the host cell containing the polynucleotide is grown, or the polynucleotide can be expressed as an intracellular polypeptide by deleting the leader or other peptides, in which case the polypeptide of the present invention or analog thereof is isolated from the host cells. The polypeptide of the present invention or analog thereof are then purified by standard protein purification methods known in the art.

The polypeptide of the present invention, analogs, or derivatives thereof can also be provided to the tissue of interest by transferring an expression vector comprising an isolated polynucleotide encoding the polypeptide of the present invention, or analog thereof to cells associated with the tissue of interest. The cells produce the peptide such that it is suitably provided to the cells within the tissue to exert a biological activity such as, for example, to reduce or inhibit inflammatory processes within the tissue of interest.

The expression vector according to the principles of the present invention further comprises a promoter. In the context of the present invention, the promoter must be able to drive the expression of the peptide within the cells. Many viral promoters are appropriate for use in such an expression vector (e.g., retroviral ITRs, LTRs, immediate early viral promoters (IEp) (such as herpes virus IEp (e.g., ICP4-IEp and ICPO-IEp) and cytomegalovirus (CMV) IEp), and other viral promoters (e.g., late viral promoters, latency-active promoters (LAPs), Rous Sarcoma Virus (RSV) promoters, and Murine Leukemia Virus (MLV) promoters). Other suitable promoters are eukaryotic promoters, which contain enhancer sequences (e.g., the rabbit β-globin regulatory elements), constitutively active promoters (e.g., the β-actin promoter, etc.), signal and/or tissue specific promoters (e.g., inducible and/or repressible promoters, such as a promoter responsive to TNF or RU486, the metallothionine promoter, etc.), and tumor-specific promoters.

Within the expression vector, the polynucleotide encoding the polypeptide of the present invention, or analog thereof and the promoter are operably linked such that the promoter is able to drive the expression of the polynucleotide. As long as this operable linkage is maintained, the expression vector can include more than one gene, such as multiple genes separated by internal ribosome entry sites (IRES). Furthermore, the expression vector can optionally include other elements, such as splice sites, polyadenylation sequences, transcriptional regulatory elements (e.g., enhancers, silencers, etc.), or other sequences.

The expression vectors are introduced into the cells in a manner such that they are capable of expressing the isolated polynucleotide encoding the polypeptide of the present invention or analog thereof contained therein. Any suitable vector can be so employed, many of which are known in the art. Examples of such vectors include naked DNA vectors (such as oligonucleotides or plasmids), viral vectors such as adeno-associated viral vectors (Berns et al, 1995, Ann. N.Y. Acad. Sci. 772:95-104, the contents of which are hereby incorporated by reference in their entirety), adenoviral vectors, herpes virus vectors (Fink et al, 1996, Ann. Rev. Neurosci. 19:265-287), packaged amplicons (Federoff et al, 1992, Proc. Natl. Acad. Sci. USA 89: 1636-1640, the contents of which are hereby incorporated by reference in their entirety), papilloma virus vectors, picomavirus vectors, polyoma virus vectors, retroviral vectors, SV40 viral vectors, vaccinia virus vectors, and other vectors. Additionally, the vector can also include other genetic elements, such as, for example, genes encoding a selectable marker (e.g., β-gal or a marker conferring resistance to a toxin), a pharmacologically active protein, a transcription factor, or other biologically active substance.

Methods for manipulating a vector comprising an isolated polynucleotide are well known in the art (e.g., Sambrook et al., 1989, Molecular Cloning: A Laboratory Manual, 2d edition, Cold Spring Harbor Press, the contents of which are hereby incorporated by reference in their entirety) and include direct cloning, site specific recombination using recombinases, homologous recombination, and other suitable methods of constructing a recombinant vector. In this manner, an expression vector can be constructed such that it can be replicated in any desired cell, expressed in any desired cell, and can even become integrated into the genome of any desired cell.

The expression vector comprising the polynucleotide of interest is introduced into the cells by any means appropriate for the transfer of DNA into cells. Many such methods are well known in the art (e.g., Sambrook et al, supra; see also Watson et al, 1992, Recombinant DNA, Chapter 12, 2d edition, Scientific American Books, the contents of which are hereby incorporated by reference in their entirety). Thus, in the case of prokaryotic cells, vector introduction can be accomplished, for example, by electroporation, transformation, transduction, conjugation, or mobilization. For eukaryotic cells, vectors can be introduced through the use of, for example, electroporation, transfection, infection, DNA coated microprojectiles, or protoplast fusion. Examples of eukaryotic cells into which the expression vector can be introduced include, but are not limited to, ovum, stem cells, blastocytes, and the like.

Cells, into which the polynucleotide has been transferred under the control of an inducible promoter if necessary, can be used as transient trans formants. Such cells themselves may then be transferred into a subject for therapeutic benefit therein. Thus, the cells can be transferred to a site in the subject such that the peptide of the invention is expressed therein and secreted therefrom and thus reduces or inhibits, for example, T cell mediated processes so that the clinical condition of the subject is improved. Alternatively, particularly in the case of cells to which the vector has been added in vitro, the cells can first be subjected to several rounds of clonal selection (facilitated usually by the use of a selectable marker sequence in the vector) to select for stable transformants. Such stable transformants are then transferred to a subject, preferably a human, for therapeutic benefit therein.

Within the cells, the polynucleotide encoding the peptides of the present invention, or analog thereof is expressed, and optionally is secreted. Successful expression of the polynucleotide can be assessed using standard molecular biology techniques (e.g., Northern hybridization, Western blotting, immunoprecipitation, enzyme immunoassay, etc.).

The present invention encompasses transgenic animals comprising an isolated to polynucleotide encoding the peptides of the invention.

### Pharmaceutical Compositions

In some embodiments, there is provided compositions (i.e., pharmaceutical compositions) comprising as an active ingredient a therapeutically effective amount of an amino acid molecule (i.e., polypeptides) of the present invention (e.g., SEQ ID NO: 1-23), and a pharmaceutically acceptable carrier.

The pharmaceutical compositions of the invention can be formulated in the form of a pharmaceutically acceptable salt of the polypeptides of the invention or their analogs, or derivatives thereof. Pharmaceutically acceptable salts include those salts formed with free amino groups such as salts derived from non-toxic inorganic or organic acids such as hydrochloric, phosphoric, acetic, oxalic, tartaric acids, and the like, and those salts formed with free carboxyl groups such as salts derived from non-toxic inorganic or organic bases such as sodium, potassium, ammonium, calcium, ferric hydroxides, isopropylamine, triethylamine, 2-ethylamino ethanol, histidine, procaine, and the like. In one embodiment, pharmaceutical compositions of the present invention are manufactured by processes well known in the art, e.g., by means of conventional mixing, dissolving, granulating, dragee-making, levigating, emulsifying, encapsulating, entrapping or lyophilizing processes.

The term “pharmaceutically acceptable” means suitable for administration to a subject, e.g., a human. For example, the term “pharmaceutically acceptable” can mean approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic compound is administered. Such pharmaceutical carriers can be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; and agents for the adjustment of tonicity such as sodium chloride or dextrose are also envisioned. The carrier may constitute, in total, from about 0.1% to about 99.99999% by weight of the pharmaceutical compositions presented herein.

The compositions can take the form of solutions, suspensions, emulsions, tablets, pills, capsules, powders, gels, creams, ointments, foams, pastes, sustained-release formulations and the like. The compositions can be formulated as a suppository, with traditional binders and carriers such as triglycerides, microcrystalline cellulose, gum tragacanth or gelatin. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in: Remington's Pharmaceutical Sciences” by E. W. Martin, the contents of which are hereby incorporated by reference herein. Such compositions will contain a therapeutically effective amount of the peptide of the invention, preferably in a substantially purified form, together with a suitable amount of carrier so as to provide the form for proper administration to the subject.

An embodiment of the invention relates to a polypeptide presented in unit dosage form and are prepared by any of the methods well known in the art of pharmacy. In an embodiment of the invention, the unit dosage form is in the form of a tablet, capsule, lozenge, wafer, patch, ampoule, vial or pre-filled syringe. In addition, in vitro assays may optionally be employed to help identify optimal dosage ranges. The precise dose to be employed in the formulation will also depend on the route of administration, and the nature of the disease or disorder, and should be decided according to the judgment of the practitioner and each patient's circumstances. Effective doses can be extrapolated from dose-response curves derived from in-vitro or in-vivo animal model test bioassays or systems.

Depending on the location of the tissue of interest, the polypeptides of the present invention can be supplied in any manner suitable for the provision of the peptide to cells within the tissue of interest. Thus, for example, a composition containing the polypeptides can be introduced, for example, into the systemic circulation, which will distribute said peptide to the tissue of interest. Alternatively, a composition can be applied topically to the tissue of interest (e.g., injected, or pumped as a continuous infusion, or as a bolus within a tissue, applied to all or a portion of the surface of the skin, etc.).

In an embodiment of the invention, polypeptides are administered via oral, rectal, vaginal, topical, nasal, ophthalmic, transdermal, subcutaneous, intramuscular, intraperitoneal or intravenous routes of administration. The route of administration of the pharmaceutical composition will depend on the disease or condition to be treated. Suitable routes of administration include, but are not limited to, parenteral injections, e.g., intradermal, intravenous, intramuscular, intralesional, subcutaneous, intrathecal, and any other mode of injection as known in the art. Although the bioavailability of peptides administered by other routes can be lower than when administered via parenteral injection, by using appropriate formulations it is envisaged that it will be possible to administer the compositions of the invention via transdermal, oral, rectal, vaginal, topical, nasal, inhalation and ocular modes of treatment. In addition, it may be desirable to introduce the pharmaceutical compositions of the invention by any suitable route, including intraventricular and intrathecal injection; intraventricular injection may be facilitated by an intraventricular catheter, for example, attached to a reservoir. Pulmonary administration can also be employed, e.g., by use of an inhaler or nebulizer.

For topical application, a peptide of the present invention, derivative, analog or a fragment thereof can be combined with a pharmaceutically acceptable carrier so that an effective dosage is delivered, based on the desired activity. The carrier can be in the form of, for example, and not by way of limitation, an ointment, cream, gel, paste, foam, aerosol, suppository, pad or gelled stick.

For oral applications, the pharmaceutical composition may be in the form of tablets or capsules, which can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate; or a glidant such as colloidal silicon dioxide. When the dosage unit form is a capsule, it can contain, in addition to materials of the above type, a liquid carrier such as fatty oil. In addition, dosage unit forms can contain various other materials which modify the physical form of the dosage unit, for example, coatings of sugar, shellac, or other enteric agents. The tablets of the invention can further be film coated.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes.

The compositions of the present invention are generally administered in the form of a pharmaceutical composition comprising at least one of the active components of this invention together with a pharmaceutically acceptable carrier or diluent. Thus, the compositions of this invention can be administered either individually or together in any conventional oral, parenteral or transdermal dosage form.

Pharmaceutical compositions according to embodiments of the invention may contain 0.1%-95% of the active components(s) of this invention, preferably 1%-70%. In any event, the composition or formulation to be administered may contain a quantity of active components according to embodiments of the invention in an amount effective to treat the condition or disease of the subject being treated.

The compositions also comprise preservatives, such as benzalkonium chloride and thimerosal and the like; chelating agents, such as EDTA sodium and others; buffers such as phosphate, citrate and acetate; tonicity agents such as sodium chloride, potassium chloride, glycerin, mannitol and others; antioxidants such as ascorbic acid, acetylcystine, sodium metabisulfote and others; aromatic agents; viscosity adjustors, such as polymers, including cellulose and derivatives thereof; and polyvinyl alcohol and acid and bases to adjust the pH of these aqueous compositions as needed. The compositions may also comprise local anesthetics or other actives.

In addition, the compositions may further comprise binders (e.g. acacia, cornstarch, gelatin, carbomer, ethyl cellulose, guar gum, hydroxypropyl cellulose, hydroxypropyl methyl cellulose, povidone), disintegrating agents (e.g. cornstarch, potato starch, alginic acid, silicon dioxide, croscarmelose sodium, crospovidone, guar gum, sodium starch glycolate), buffers (e.g., Tris-HCl, acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g. sodium lauryl sulfate), permeation enhancers, solubilizing agents (e.g., glycerol, polyethylene glycerol), anti-oxidants (e.g., ascorbic acid, sodium metabisulfite, butylated hydroxyanisole), stabilizers (e.g. hydroxypropyl cellulose, hyroxypropylmethyl cellulose), viscosity increasing agents (e.g. carbomer, colloidal silicon dioxide, ethyl cellulose, guar gum), sweeteners (e.g. aspartame, citric acid), preservatives (e.g., Thimerosal, benzyl alcohol, parabens), lubricants (e.g. stearic acid, magnesium stearate, polyethylene glycol, sodium lauryl sulfate), flow-aids (e.g. colloidal silicon dioxide), plasticizers (e.g. diethyl phthalate, triethyl citrate), emulsifiers (e.g. carbomer, hydroxypropyl cellulose, sodium lauryl sulfate), polymer coatings (e.g., poloxamers or poloxamines), coating and film forming agents (e.g. ethyl cellulose, acrylates, polymethacrylates) and/or adjuvants.

The polypeptides of the present invention, derivatives, or analogs thereof can be delivered in a controlled release system. Thus, an infusion pump can be used to administer the peptide such as the one that is used, for example, for delivering insulin or chemotherapy to specific organs or tumors. In one embodiment, the peptide of the invention is administered in combination with a biodegradable, biocompatible polymeric implant, which releases the peptide over a controlled period of time at a selected site. Examples of preferred polymeric materials include, but are not limited to, polyanhydrides, polyorthoesters, polyglycolic acid, polylactic acid, polyethylene vinyl acetate, copolymers and blends thereof (See, Medical applications of controlled release, Langer and Wise (eds.), 1974, CRC Pres., Boca Raton, Fla., the contents of which are hereby incorporated by reference in their entirety). In yet another embodiment, a controlled release system can be placed in proximity to a therapeutic target, thus requiring only a fraction of the systemic dose.

In one embodiment, compositions of the present invention are presented in a pack or dispenser device, such as an FDA approved kit, which contain one or more unit dosage forms containing the active ingredient. In one embodiment, the pack or dispenser device is accompanied by instructions for administration.

In one embodiment, it will be appreciated that the polypeptides of the present invention can be provided to the individual with additional active agents to achieve an improved therapeutic effect as compared to treatment with each agent by itself. In another embodiment, measures (e.g., dosing and selection of the complementary agent) are taken to adverse side effects which are associated with combination therapies.

A “therapeutically effective amount” of the peptide is that amount of peptide which is sufficient to provide a beneficial effect to the subject to which the peptide is administered. More specifically, a therapeutically effective amount means an amount of the peptide effective to prevent, alleviate or ameliorate tissue damage or symptoms of a disease of the subject being treated.

In some embodiments, preparation of effective amount or dose can be estimated initially from in vitro assays. In one embodiment, a dose can be formulated in animal models and such information can be used to more accurately determine useful doses in humans.

In one embodiment, toxicity and therapeutic efficacy of the active ingredients described herein can be determined by standard pharmaceutical procedures in vitro, in cell cultures or experimental animals. In one embodiment, the data obtained from these in vitro and cell culture assays and animal studies can be used in formulating a range of dosage for use in human. In one embodiment, the dosages vary depending upon the dosage form employed and the route of administration utilized. In one embodiment, the exact formulation, route of administration and dosage can be chosen by the individual physician in view of the patient's condition. [See e.g., Fingl, et al., (1975) “The Pharmacological Basis of Therapeutics”, Ch. 1 p. 1].

In one embodiment, depending on the severity and responsiveness of the condition to be treated, dosing can be of a single or a plurality of administrations, with course of treatment lasting from several days to several weeks or until cure is effected or diminution of the disease state is achieved. In one embodiment, the amount of a composition to be administered will, of course, be dependent on the subject being treated, the severity of the affliction, the manner of administration, the judgment of the prescribing physician, etc. In one embodiment, compositions including the preparation of the present invention formulated in a compatible pharmaceutical carrier are also prepared, placed in an appropriate container, and labeled for treatment of an indicated condition.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

### Therapeutic Use

According to another aspect, there is provided a method for treating a mesotrypsin-associated and/or kallikrein-6 associated pathological condition in a subject in need thereof, the method comprising the step of administering to said subject a pharmaceutical composition comprising an effective amount of an isolated polypeptide comprising the amino acid selected from the group consisting of SEQ ID NO: 1-23, and a pharmaceutically acceptable carrier, thereby treating the mesotrypsin-associated and/or kallikrein-6 associated pathological condition in a subject in need thereof. In some embodiments, the mesotrypsin-associated and/or kallikrein-6 associated pathological condition is a cancer.

According to another aspect, the invention provides a pharmaceutical composition comprising an effective amount of an isolated polypeptide comprising the amino acid selected from the group consisting of SEQ ID NO: 1-23, and a pharmaceutically acceptable carrier, for use in treating mesotrypsin-associated and/or kallikrein-6 associated pathological condition in a subject in need thereof. In some embodiments, the mesotrypsin-associated and/or kallikrein-6 associated pathological condition is a cancer.

According to another aspect, the invention provides use of a pharmaceutical composition comprising an effective amount of an isolated polypeptide comprising the amino acid selected from the group consisting of SEQ ID NO: 1-23 and a pharmaceutically acceptable carrier, for preparation of a medicament for treating a mesotrypsin-associated and/or kallikrein-6 associated pathological condition in a subject in need thereof. In some embodiments, the medicament for treating cancer in a subject in need thereof.

According to another embodiment, the pharmaceutical composition comprises an effective amount of an isolated polypeptide comprising the amino acid selected from the group consisting of SEQ ID NO: 1-14, and a pharmaceutically acceptable carrier.

According to another embodiment, the cancer is a mesotrypsin-associated cancer. According to another embodiment, said cancer is selected from the group consisting of prostate, lung, colon, breast, pancreas, gastric and non-small cell lung cancer (NSCLC) or metastasis thereof. According to another embodiment, said cancer is prostate cancer. According to another embodiment, said cancer is gastric cancer.

According to another embodiment, said treating is inhibiting invasiveness of a cancerous cell.

### Diagnostic Use

According to some aspects, the APPI variants of the invention may be utilized as affinity agents for the detection and/or analysis of mesotrypsin and/or kallikrein-6. The term “affinity agent” generally refers to a molecule that specifically binds to an antigen (e.g., mesotrypsin, kallikrein-6).

In some embodiments, the APPI variants of the invention are labeled. Non-limiting examples of labels are fluorescent labels for fluorescence microscopy, radioactive labels for autoradiography, or electron dense for electron microscopy. The labeled APPI variant may be used essentially in the same type of applications as labeled monoclonal antibodies, e.g. fluorescence and radio assays, cytofluorimetry, fluorescence activated cell sorting etc. The principles of such techniques can be found in immunochemistry handbooks, for example: A Johnstone and R Thorpe, Immunochemistry in practice, 2^{nd} Edition (1987), blackwell Scientific publications, Oxford London Edinburgh Boston Palo Alto Melbourne.

According to some aspects, the invention provides a method for directly visualizing the cellular distribution of mesotrypsin and/or kallikrein-6. In some embodiments, the method comprises the step of contacting a cell with a labeled APPI variants of the invention. In some embodiments, the method further includes a step of imaging the cell. In some embodiments, the cell is a whole cell, a population of cells, cells fixed onto slides or sections through solid tissue. In some embodiments, the contacting is performed in-vitro. In other embodiments, the contacting is performed in vivo.

In some embodiments, there is provided an imaging reagent composed of the peptide of the invention (i.e., the APPI variant described herein) as an affinity agent coupled, directly or indirectly, to an imaging agent. In one embodiment, said imaging reagent is predictive of a mesotrypsin-associated disease or a disease state. In another embodiment, said mesotrypsin-associated disease or disorder is cancer such as prostate cancer. In one embodiment, the imaging reagent is predictive of a kallikrein-6-associated disease or a disease state.

A “disease state” refers to the current status of a disease which may have been previously diagnosed, such prognosis, risk-stratification, assessment of ongoing drug therapy, prediction of outcomes, determining response to therapy, diagnosis of a disease or disease complication, following progression of a disease or providing any information relating to a patient's health status over time.

In one embodiment, said imaging agent is an isotope. Typically, useful diagnostic isotopes (e.g., for PET and SPECT-based detection and imaging) for use in accordance with the present invention include: ¹⁸F, ⁴⁷Sc, ⁵¹Cr, ⁵²Fe, ^{52m}Mn, ⁵⁶Ni, ⁵⁷Ni, ⁶²Cu, ⁶⁴Cu, ⁶⁷Ga, ⁶⁸Ga, ⁷²As, ⁷⁵Br, ⁷⁶Br, ⁷⁷Br, ⁸²Br, ⁸⁹Zr, ^{94m}Tc, ⁹⁷Ru, ^{99m}Tc, ¹¹¹In, ¹²³I, ¹²⁴I, ¹³¹I, ¹⁹¹Pt, ¹⁹⁷Hg, ²⁰¹Tl, ²⁰³Pb, ^{101m}In, ¹²⁰I.

In another embodiment, the invention provides a method of imaging a neoplastic tissue, the method comprises administering to a subject having (or suspected of having) a neoplasia, an imaging reagent compound of the invention, and detecting the compound following distribution thereof in vivo. In some embodiments, said method of imaging includes the subsequent step (e.g., following the detection step) of generating an image of the detected distributed compound. The detection step may be performed using PET or single photon emission computed tomography (SPECT) when the label is a radionuclide. When magnetic or paramagnetic labels are employed, magnetic resonance imaging may be used.

In another embodiment, the present invention provides a kit comprising:
a. an APPI variant of the invention or an analog, a derivative or fragment thereof, or a composition comprising said APPI variant; and
b. at least one signal producing label.

In some embodiments, the APPI variant of said kit is conjugated, directly or indirectly, to the signal-producing label, such as a tag, as described herein.

In some embodiments, the kit is for assessing mesotrypsin function in a cell. In some embodiments, the kit is for assessing kallikrein-6 function in a cell. In some embodiments, the kit is for diagnosing a mesotrypsin associated pathological condition in a subject in need thereof. In some embodiments, the kit is for diagnosing a kallikrein-6 associated pathological condition in a subject in need thereof. In some embodiments, the kit is for diagnosing cancer in a subject in need thereof.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

In the description and claims of the present application, each of the verbs, “comprise,” “include” and “have” and conjugates thereof, are used to indicate that the object or objects of the verb are not necessarily a complete listing of components, elements or parts of the subject or subjects of the verb.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

### Plasmids and Cell Culture

Cells.

PC3-M cells were maintained in RPMI 1640 (Invitrogen) supplemented with 10% fetal bovine serum (Invitrogen).

Reagents.

Synthetic oligonucleotides were obtained from Integrated DNA Technologies. Restriction enzymes and polymerases were purchased from New England Biolabs, and dNTPs, from Jena Bioscience. Bacterial plasmid extraction and purification kits were obtained from RBC Bioscience, and yeast plasmid extraction kits, from Zymo Research. The methylotrophic yeast Pichia pastoris strain GS115, Pichia expression vector (pPIC9K), and fluorescein (FITC)-conjugated streptavidin were obtained from Invitrogen. Bovine trypsin, phycoerytherin (PE)-conjugated anti mouse antibody, and the substrates benzyloxycarbonyl-Gly-Pro-Arg-p-nitroanalide (Z-GPR-pNA), 4-nitrophenyl 4-guanidinobenzoate (pNPGB), and benzoyl-L-arginine-p-nitroanilide (L-BAPA) were obtained from Sigma-Aldrich. Mouse anti-c-Myc antibody (Ab-9E10) was obtained from Abcam. EZ-Link NHS-PEG4 biotinylation kit was purchased from ThermoFisher Scientific. Factor-XIa and its substrate S-2366 (Chromogenix) were obtained from Hematologic Technologies Inc. and Diapharma, respectively.

Synthesis and Cloning of the DNA Encoding APPI_{WT}.

The inhibitor domain of the amyloid precursor protein (APPI_{WT}) gene was constructed based on a published sequence (PDB id IZJD) by using codons optimized for both Saccharomyces cerevisiae and P. pastoris usage and synthesized by PCR-assembly using six overlapping oligonucleotides. The final PCR assembled fragment was gel-purified and cloned into the YSD vector (pCTCON) via transformation of EBY100 yeast cell with linearized vector (digested with NheI and BamHI) and the PCR product. This simultaneous cloning and transformation occurs via the in vivo homologous recombination between the vector and the PCR insert to generate the YSD plasmid. After sequence verification, the DNA construct served as the template for combinatorial library generation. The individual YSD APPI mutants were prepared by the same methodology.

Generation of a Combinatorial Based APPI Library.

Generation of the YSD APPI library is described in detail below. In brief, a randomly mutated version of the APPI gene was first constructed by error-prone PCR using nucleotide analogues and low-fidelity Taq polymerase. The resulting insert was amplified and transformed into yeast through homologous recombination. Random mutagenesis in the APPI sequence generated an APPI library with 0-3 mutations per clone, yielding an experimental library size of about 9×10⁶ clones.

Flow Cytometry and Cell Sorting.

Yeast-displayed APPI library and individual APPI variants were grown in SDCAA selective medium (2% dextrose, 1.47% sodium citrate, 0.429% citric acid monohydrate, 0.67% yeast nitrogen base and 0.5% casamino acids) and induced for expression with galactose medium (same as for SDCAA, but with galactose instead of dextrose), according to established protocols. Due to the different enzymatic turnover times of APPI and its variants by the target trypsins, i.e., bovine trypsin or mesotrypsin, two methods for trypsin-labeling were used, namely, ‘double staining’ and ‘triple staining’, for the detection of proteolytically resistant clones, as described below. In the first step of labeling, approximately 1×10⁶ cells were labeled with the appropriate catalytically active trypsin and a 1:50 dilution of mouse anti-c-Myc antibody in trypsin buffer (TB; 100 mM Tris-HCl, pH 8.0, 1 mM CaCl₂) supplemented with 1% bovine serum albumin (BSA) for 30 min at room temperature. In the second step of labeling, for ‘double staining’ the cells were exposed to biotinylated-bovine trypsin or mesotrypsin, and for ‘triple staining’ the cells were treated with non-biotinylated bovine trypsin or mesotrypsin. For ‘triple staining’, a third labelling step was then applied: the cells were washed with TB and incubated with 2 μM of biotinylated catalytically inactive mesotrypsin-S195A for 1 h at room temperature. Finally, for both ‘double staining’ and ‘triple staining’, cells were washed with ice-cold TB followed by incubation with a 1:800 dilution of fluorescein (FITC)-conjugated streptavidin and a 1:50 dilution of PE-conjugated anti mouse secondary antibody for 30 min on ice. Cells were washed again and analyzed by dual-color flow cytometry (Accuri C6; BD Biosciences).

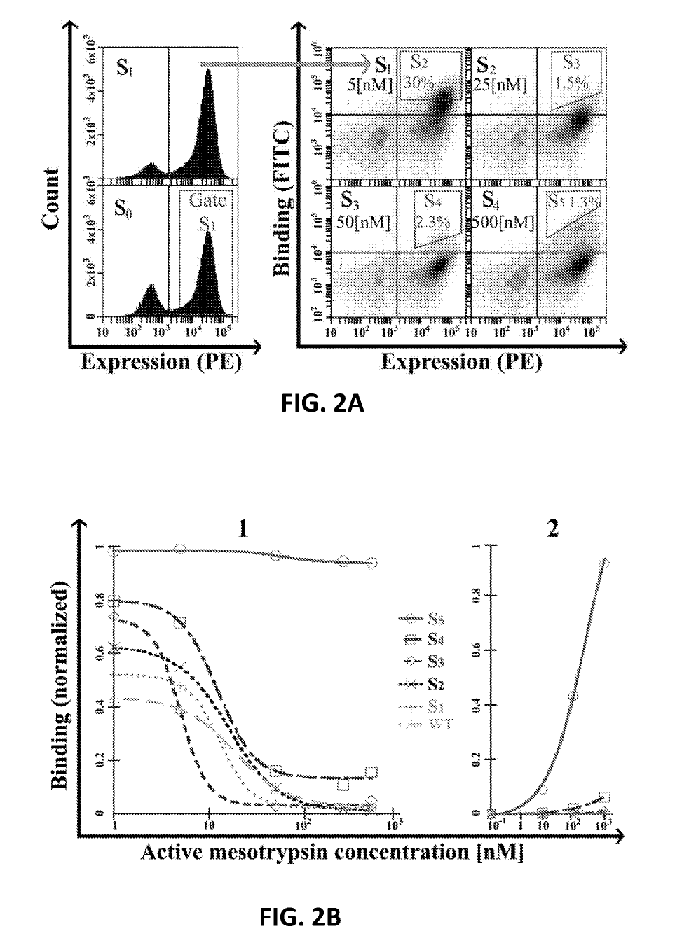

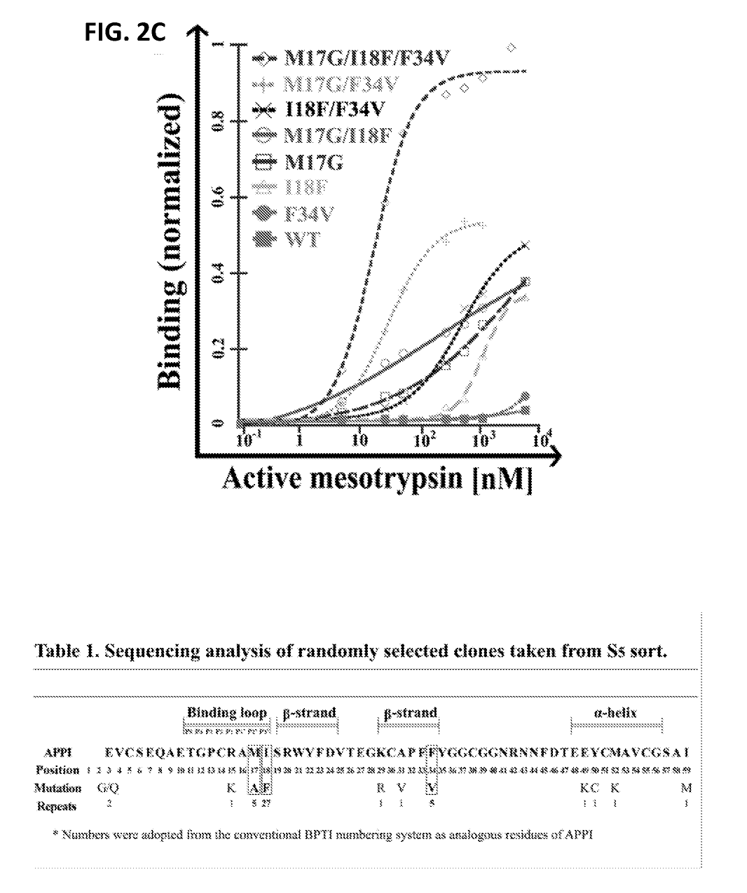

Cell sorting of ‘triple’-stained cells was carried out as described in FIG. 2A with a iCyt Synergy FACS. In brief, approximately 1×10⁸ cells were first sorted to select for high expressing clones (c-Myc clear). Sorted cells were then grown in selective medium, and several colonies were sequenced. Following each triple staining sort, the number of yeast cells used for subsequent sorting was at least 10-fold in excess of the number of sorted cells. Several clones from each round of sorting were sequenced. The concentration of the target protein in each sort is shown in FIG. 2A.

Production of Recombinant Proteins.

Recombinant human anionic trypsinogen, human cationic trypsinogen and human mesotrypsinogen, in addition to the catalytically inactive S195A mutant of mesotrypsinogen, were expressed in E. coli, extracted from inclusion bodies, refolded, purified and activated with bovine enteropeptidase as described in previous work (alameh, M. A., et al., J Biol Chem, 2008. 283(7): p. 4115-23; Salameh, M. A., et al., Biochem J, 2011. 440(1): p. 95-105). Mesotrypsin and mesotrypsin-S195A were biotinylated for use in YSD screens, and biotin incorporation quantified by 4′-hydroxyazobenzene-2-carboxylic acid (HABA) assay, using the EZ-Link NHS-PEG4 biotinylation kit (ThermoFisher Scientific) according to manufacturer instructions. Constructs, cloning, expression and purification of APPI variants are described in detail below. In brief, APPI variants were expressed in P. pastoris strain GS115 under control of the AOX1 (alcohol oxidase) promoter using the expression vector pPIC9K. Inhibitors were purified from the yeast culture supernatant by immobilized metal affinity chromatography using a HisTrap 5-ml column (GE Healthcare). Eluted inhibitors were concentrated, and the buffer was replaced with TB. Gel filtration chromatography was performed on a 16/600 Superdex 75 column (GE Healthcare) equilibrated with TB at a flow rate of 1 ml/min on an ÄKTA pure instrument (GE Healthcare). Purification yields for all APPI variants were 5-20 mg per one-liter culture flask.

Trypsin Inhibition Studies.

The concentrations of mesotrypsin, cationic trypsin, anionic trypsin and bovine trypsin were quantified by active site titration using pNPGB, which serves as both irreversible trypsin inhibitor and substrate. Concentrations of FXIa and Kallikrein-6 were determined by UV-Vis absorbance at 280 nm with extinction coefficient (ε₂₈₀) of 214.4×10³ M⁻¹ cm⁻¹ and 34.67×10³ M⁻¹ cm⁻¹, respectively. Concentrations of the chromogenic substrates Z-GPR-pNA and S-2366 were determined by an end-point assay (from the change in the absorbance (plateau after complete hydrolysis) that is obtained by the release of p-nitroaniline). Concentrations of APPI variants were determined by titration with pre-titrated bovine trypsin and the substrate L-BAPA, as previously described (Salameh, M. A., et al., Protein Sci, 2012. 21(8): p. 1103-12.).

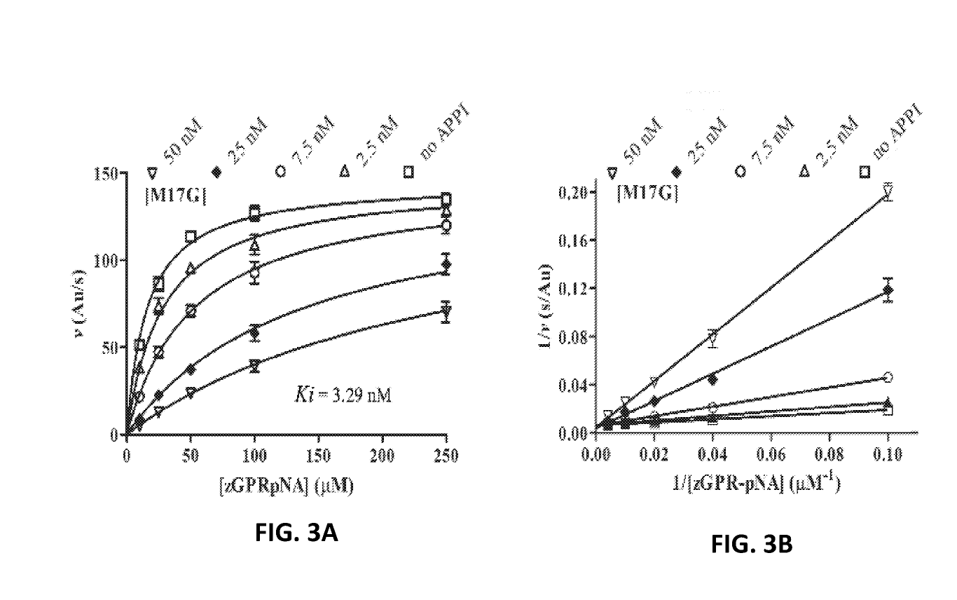

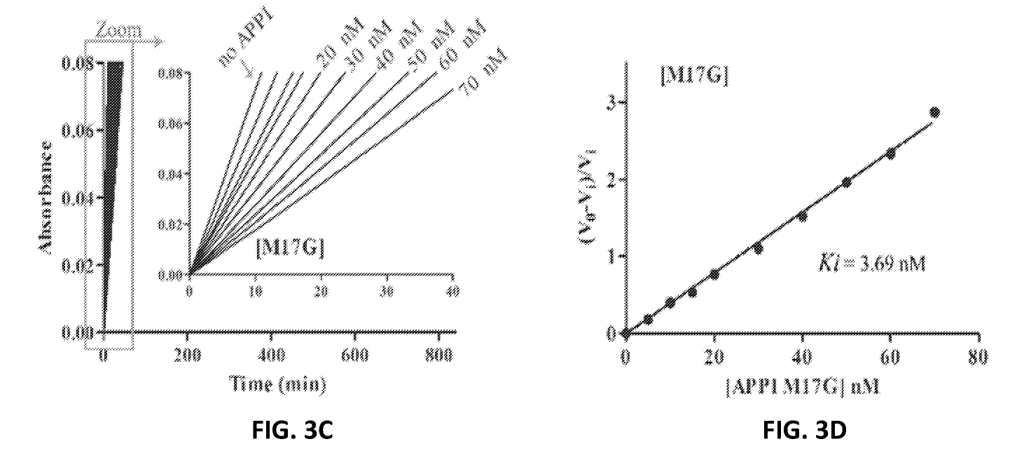

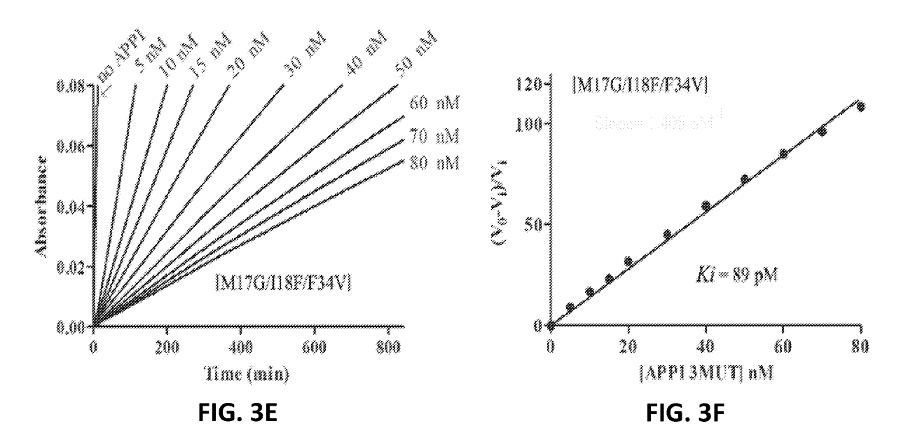

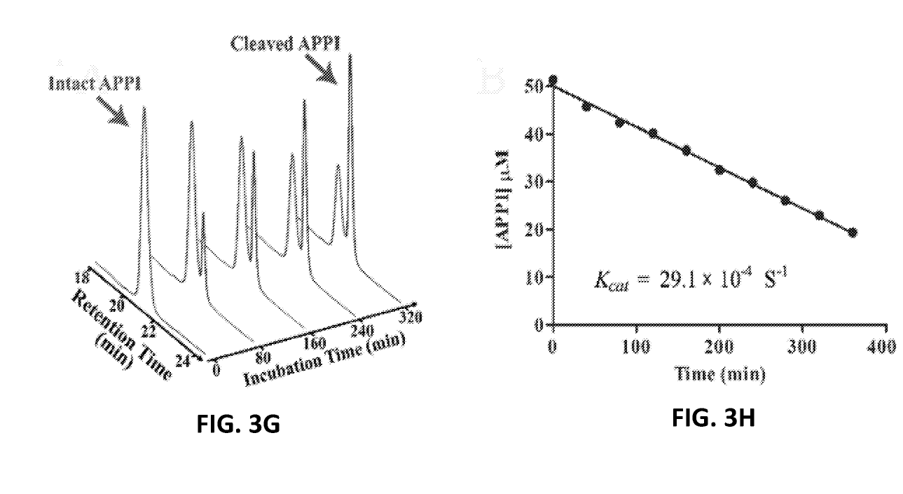

The constant Kᵢ of APPI_{WT} and its variants: APPI_{M17G}, APPI_{I18F} and APPI_{F34V} in complex with mesotrypsin were determined according to the previously described methodology with minor changes (Salameh, M. A., et al., 2012, ibid). Later, this methodology was adjusted for measuring the dissociation constant of APPI_{M17G/I18F/F34V} in complex with FXIa. Briefly, stock solutions of enzyme, substrate, and APPI proteins were prepared at 40×the desired final concentrations. Assays were performed at 37° C. in the presence of different concentrations of substrate and inhibitor in a Synergy2 microplate spectrophotometer (BioTek). The concentrations of reagents are given in FIGS. 3A and 3B. Assay buffer (296 μl), substrate (8 μl), and APPI (8 μl) were mixed and equilibrated in 96-well microplate (Greiner) prior to the addition of enzyme (8 μl from 10 nM mesotrypsin or 5 nM FXIa). Here, ‘assay buffer’ represents TB or FXIa buffer (FB; 50 mM Tris-HCl, pH 7.6, 150 mM NaCl, 5 mM CaCl2) and 0.1% BSA) whereas ‘substrate’ represents Z-GPR-pNA or S-2366 for mesotrypsin or FXIa, respectively. Reactions were followed spectroscopically for 5 min, and initial rates were determined from the increase in absorbance caused by the release of p-nitroaniline (ε₄₁₀=8480 M⁻¹ cm⁻¹). Data were globally fitted by multiple regression to Equation 1, the classic competitive inhibition equation, using Prism (GraphPad Software, San Diego Calif.). It should be noted that Equation 1 assumes that the inhibitor concentration is not significantly reduced by its binding with the enzyme, therefore, making it appropriate for measuring the dissociation constants for only weak interactions. Although the dissociation constants calculated using Equation 1 are relatively high (i.e. weak interactions; Table 2), inhibitor concentrations that were at least 10 times in access over the enzyme (i.e. any reduction of the inhibitor concentration upon binding is therefore negligible) were used. Reported inhibition constants are average values obtained from three independent experiments.

$$V = \frac{{{K}_{cat} [ E ]}_{0} [ S ]}{{K}_{m} ( 1 + [ I ] / {K}_{i} ) + [ S ]} \qquad ( Eq . 1 )$$

Inhibition studies of (i) mesotrypsin with APPI variants M17G, M17G/I18F, M17G/F34V, I18F/F34V and M17G/I18F/F34V, (ii) cationic trypsin, anionic trypsin and Kallikrein-6 with APPI_{WT} and APPI_{M17G/I18F/F34V}, and (iii) FXIa with APPI_{M17G/I18F/F34V}, were carried out in a similar manner, but the finding of slow, tight binding behavior required a different kinetic treatment as compared to the one presented in Eq. 1. In tight binding kinetics, the reduction of the inhibitor concentration upon binding is significant (i.e. tight binding/strong interactions) and should be considered. Briefly, tight binding experiments including the reactions of mesotrypsin, cationic trypsin and anionic trypsin were conducted at fixed concentration of Z-GPR-pNA (145 μM), the inhibitor concentrations ranged between 5-80 nM, and the enzyme concentration was 0.1 nM (FIG. 3C-3F). Enzyme (8 μl), inhibitor (8 μl) and TB (144 μl) were pre-incubated at room temperature for 20-60 min; the reactions were then initiated by dilution of the enzyme/inhibitor mixture into a pre-equilibrated microplate (non-binding, 96 well; Greiner) containing TB (152 μl) and substrate (8 μl). The microplates were covered with lids and sealed with Parafilm to prevent evaporation. Reactions were run at 25° C. and were followed spectroscopically for 14 h so that reliable steady-state rates could be obtained. Conversion of substrate to product did not exceed 10% over the reaction time course.

Tight binding reactions of FXIa and Kallikrein-6 were carried out in the same manner with minor changes as follows: for FXIa the substrate (S-2366) concentration was 600 μM, inhibitor concentrations ranged between 2-10 nM, enzyme concentration was 0.125 nM, assay buffer was FB, and the reactions were run at 37° C. and followed spectroscopically for 1 h. Reactions of Kallikrein-6 were carried out at fixed concentration of BOC-Phe-Ser-Arg-AMC (1 mM), the inhibitor concentrations ranged between 5-50 nM, enzyme concentration was 1 nM, assay buffer was Kallikrein buffer (KB; 50 mM Tris-HCl, pH 7.3, 100 mM NaCl and 0.2% BSA) and the reactions were run at 37° C. (for 5 h) and followed by fluorescent signal in a Tecan Infinite 200 PRO NanoQuant microplate reader set at 355 nm for excitation and 460 nm for emission.

Inhibition constants for tight binding reactions were calculated using Equation 2, as described previously (Salameh, M. A., et al., 2012, ibid.), where vᵢ and v₀ are the steady-state rates in the presence and absence of inhibitor, K_{M} is the Michaelis constant for substrate cleavage, and [S]₀ and [I]₀ are the initial concentrations of substrate and inhibitor, respectively. Calculations were performed using K_{M} values of 24.66±1.3 μM for mesotrypsin, 22.84±1.9 μM for cationic trypsin, 10.69±0.65 μM for anionic trypsin, 361.3±12.1 μM for FXIa, and 329.3±2.5 μM for Kallikrein-6 as determined from at least three Michaelis-Menten kinetic experiments.

$$\frac{( {V}_{0} - {V}_{i} )}{{V}_{i}} = \frac{{[ I ]}_{0}}{{K}_{i} ( 1 + {[ S ]}_{0} / {K}_{m} )} \qquad ( Eq . 2 )$$

Hydrolysis Studies.

The cleavage of intact APPI variants (between the residues Arg15-Ala16) in time course incubations with catalytically active mesotrypsin was monitored by HPLC as described previously, with minor modifications. Briefly, mesotrypsin was incubated with the APPI mutants in TB at 37° C.; inhibitor concentrations were 50 μM and mesotrypsin concentrations were varied from 0.05 μM to 2.5 μM. For HPLC analysis, aliquots of 30 μl were withdrawn from the hydrolysis reactions at periodic intervals (over six hours), and samples were quenched immediately by acidification with 70 μl of 0.3 M HCl. Samples were resolved on a 50×2.0-mm Jupiter 4μ 90-Å C₁₂ column (Phenomenex) with a gradient of 0-100% acetonitrile in 0.1% trifluoroacetic acid (TFA) at a flow rate of 0.6 ml/min over 50 min. Intact inhibitors were quantified by peak integration of absorbance traces monitored at 210 nm. Initial rates were obtained by linear regression using a minimum of six data points within the initial linear phase of the reaction. Hydrolysis rates reported for each inhibitor represent the average of three independent experiments.

Prostate Cancer Cell Invasion Assays.

Matrigel transwell invasion assays of PC3-M human prostate cancer cells were conducted essentially as described previously (Hockla, A., et al., Mol Cancer Res, 2012. 10(12): p. 1555-66). Cells subjected to knockdown of PRSS3 expression, using lentiviral short hairpin RNA construct NM_002771.2-454s1c1 (Sigma), served as a positive control for suppression of mesotrypsin activity in all experiments. Efficient knockdown was confirmed by qRT/PCR using an Applied Biosystems 7900HT Fast Real-Time PCR System; PRSS3 was detected using TaqMan assay Hs00605637_m1 and normalized against GAPDH expression using Taqman assay Hs99999905_m1. Cells used for all other conditions were instead transduced with a non-target control lentiviral vector containing a short hairpin that does not recognize any human genes. Prior to invasion assays, cells were seeded at 1.5×10⁶ cells per 10 cm dish (day 1), media were replaced with a mixture of 3.6 mL RPMI containing 10% FBS and 10 μg/ml polybrene and 2.4 mL conditioned lentiviral media containing lentiviral particles to transduce cells (day 2), media were changed after 24 h and cells selected with 2 μg/ml puromycin (day 3), and then cells were trypsinized, washed, and seeded into 24-well 8.0 μm cell culture inserts (BD) previously coated with 50 μg Matrigel (2×10⁴ cells per insert in 400 μl media; day 4). In some experimental conditions, APPI_{WT} or APPI_{M17G/I18F/F34V} proteins (1 nM-1 μM) were added to the cell suspensions at the time of seeding into transwell inserts; quadruple biological replicates were performed per treatment. Cells were allowed to invade toward a chemoattractant medium comprised of 750 μL NIH/3T3 cell-conditioned serum free medium (DMEM supplemented with 50 pig/mL ascorbic acid). After 18 hours (day 5), non-invading cells were removed, filters fixed with methanol, stained with crystal violet, and air dried. Stained filters were photographed and invading cells counted using Image-Pro 6.3 software (Media Cybernetics). Consistent results were obtained from 5 independent experiments.

Prostate Cancer Cell 3D Culture Assays.

PC3-M cells for 3D culture assays were transduced with either a lentiviral shRNA construct targeting PRSS3 or with a non-target control construct recognizing no human genes, following the schedule described above for Prostate cancer cell invasion assays. On day 4, cells were seeded into 3D cultures in Matrigel following the ‘on-top’ protocol essentially as described previously (Hockla, A., et al., 2012, ibid.). Briefly, in 12-well plates, a base layer of 250 μl 100% Matrigel was polymerized, PC3-M cells (2×10⁴ cells/well) in serum-free RPMI 1640 medium were seeded and allowed to attach, excess medium was aspirated, and cells were overlaid with 500 μl of medium supplemented with 10% Matrigel and 0.5% fetal bovine serum as well as with 1 nM, 10 nM, or 100 nM of APPI_{WT} or APPI_{M17G/I18F/F34V} in some conditions as indicated. Cultures were maintained at 37° C. in 5% CO₂ for 3 days, photographed, and analyzed.

Synthesis and Cloning of the DNA Encoding the APPI_{WT}.

The DNA sequence of APPI_{WT} attached to a peptide linker (NH₃⁺-APPI_{WT}-LPDKPLAFQDPS-COO⁻) was generated by PCR assembly using the following six overlapping oligonucleotides (5′-3′):

Oligo1
(SEQ ID NO: 26)
(GATGGTATTTCGATGTTACTGAAGGTAAATGTGCTCCATTCTTCTATGG
TGGTTGTGGTG);
Oligo1′
(SEQ ID NO: 27)
(CCACAAACAGCCATACAATATTCTTCAGTATCGAAATTATTTCTATTAC
CACCACAACCACCATAGAAGAAT);
Oligo2:
(SEQ ID NO: 28)
(GAAGTTTGTTCTGAACAAGCTGAAACTGGTCCATGTAGAGCTATGATTT
CTAGATGGTATTTCGATGTTACTG);
Oligo2′:
(SEQ ID NO: 29)
(GGAAAGCCAATGGTTTATCTGGCAAGGATCCAATAGCAGAACCACAAAC
AGCCATACAATATTC);
Oligo3:
(SEQ ID NO: 30)
(GGTGGTTCTGGTGGTGGTGGTTCTGGTGGTGGTGGTCTGCTAGCGAAGT
TTGTTCTGAACAAGCTG);
Oligo3′:
(SEQ ID NO: 31)
(GAGCTATTACAAGTCCTCTTCAGAAATAAGCTTTTGTTCAGATGGATCT
TGGAAAGCCAATGGTTTATC).

The synthetic insert gene was assembled by a set of three PCRs using Phusion DNA polymerase, while each paired reaction (OligoX/X′) served as a template for the following reaction.

DNA Sequence of APPI caring combination of specific mutations were generated using the same methodology, however with oligonucleotides containing the respectively mutations:

Oligo2^{a}:
(SEQ ID NO: 32)
(GAAGTTTGTTCTGAACAAGCTGAAACTGGTCCATGTAGAGCTGGTTTTT
CTAGATGGTATTTCGATGTTACTG);
Oligo2^{b}:
(SEQ ID NO: 33)
(GAAGTTTGTTCTGAACAAGCTGAAACTGGTCCATGTAGAGCTGGTATTT
CTAGATGGTATTTCGATGTTACTG);
Oligo2^{c}:
(SEQ ID NO: 34)
(GAAGTTTGTTCTGAACAAGCTGAAACTGGTCCATGTAGAGCTATGTTTT
CTAGATGGTATTTCGATGTTACTG);
Oligo1^{′a}:
(SEQ ID NO: 35)
(CCACAAACAGCCATACAATATTCTTCAGTATCGAAATTATTTCTATTAC
CACCACAACCACCATAGACGAATG).

The final PCR assembled fragment was gel-purified and subcloned into the YSD vector (pCTCON) using transformation by electroporation of EBY100 yeast cells having a linearized vector (digested with NheI and BamHI) and the PCR product. Next, plasmid DNA was extracted from the yeast clones using a Zymoprep kit and transformed into electrocompetent E. coli cells for plasmid miniprep and sequence analysis.

Generation of Combinatorial APPI Library.

After assembly and cloning of APPI_{WT}, the plasmid construct served as the template for the subsequent generation of the combinatorial library by using error-prone PCR. To generate a mutation frequency of ˜3 mutations per clone, the PCR reaction was optimized to 15×PCR doublings of the 300-bp APPI fragment (including plasmid homologue regions) with low-fidelity Taq polymerase, 1% nucleotide analogues and 2 mM MgCl₂. The resulting mutated insert was amplified and transformed into yeast through homologous recombination to generate a library of about 9×10⁶ in size, as estimated by dilution plating on selective SDCAA plates (same as for SDCAA, but supplemented with 15% agar). Sequencing results revealed an average mutation rate of 0-3 mutations per 300 bp.

Construction and Cloning of the Expression Vector pPIC9K-APPI.

The human cDNA of APPI_{WT} was amplified by PCR using Phusion DNA polymerase with an upstream primer: 5′-AGCGTATACGTAGACTATAAGGATGACGACGACAAAGAATTCGAAGTTTGTTCTGAA CAAGCTG-3′ (SEQ ID NO: 36) and a downstream primer: 5′-ATAGTTTAGCGGCCGCATGATGGTGGTGATGGTGCCTAGGAATAGCAGAACCACAAA CAGC-3′ (SEQ ID NO: 37). The resulting construct included four restriction sites and two epitope tags (FLAG and HIS₆) as follows: SnaBI-FLAG-EcoRI-APPI_{WT}-AvrII-HIS₆-NotI. The obtained DNA fragment was digested with SnaBI and NotI, and subcloned by using the same restriction sites of Pichia expression vector pPIC9K by standard methods. Next, the recombinant expression plasmid was used as a template for the construction of the APPI variants as follows: cDNA of each variant was amplified by PCR with an upstream primer: 5′-CGGAGCGAATTCGAAGTTTGTTCTGAACAAGCTG-3′ (SEQ ID NO: 38) and a downstream primer: 5′-CGCTACCCTAGGAATAGCAGAACCACAAACAGC-3′ (SEQ ID NO: 39). The resulting construct included the restriction sites EcoRI and AvrII. The obtained DNA fragment was digested with EcoRI and AvrII, and subcloned using the same restriction sites of the template vector. Finally, the sequence each of the recombinant expression plasmids was confirmed by DNA sequencing analysis.

Expression vectors were linearized by SacI digestion and used to transform P. pastoris strain GS115 by electroporation. This resulted in insertion of the construct at the AOX1 locus of P. pastoris, thereby generating a His⁺ Mut⁺ phenotype. Transformants were selected for the His⁺ phenotype on 2% agar containing regeneration dextrose biotin (RDB; 18.6% sorbitol, 2% dextrose, 1.34% yeast nitrogen base, 4×10⁻⁵ percent biotin, and 0.005% each of L-glutamic acid, L-methionine, L-lysine, L-leucine, and L-isoleucine) and allowed to grow for 2 d at 30° C. Cells were harvested from the plates and subjected to further selection for high copy number by their ability to grow on 2% agar containing 1% yeast extract, 2% peptone, 2% dextrose medium, and the antibiotic G418 (Geneticin, 4 mg/ml, Invitrogen).

To verify direct insertion of the construct at the AOX1 locus of P. pastoris, the genomic DNA of the highest APPI-expressing colony from each APPI variant was extracted and amplified by PCR with an AOX1 upstream primer: 5′-GACTGGTTCCAATTGACAAGC-3′ (SEQ ID NO: 40) and an AOX1 downstream primer: 5′-GCAAATGGCATTCTGACATCC-3′(SEQ ID NO: 41). The resulting linear DNA was gel purified and its correct sequence was confirmed by DNA sequencing analysis.

Large-Scale Expression and Purification of APPI.

GS II5-APPI clones were first inoculated into 50 mL of BMGY (1% yeast extract, 2% peptone, 0.23% potassium phosphate monobasic, 1.18% potassium phosphate dibasic, 1.34% yeast nitrogen base, 4×10⁻⁵ percent biotin and 1% glycerol) to an OD₆₀₀=10.0, followed by scale up to 500 mL of BMGY until OD₆₀₀=10.0 was reached (overnight growth at 30° C. with shaking at 300 rpm). Cells were harvested by centrifugation and resuspended in 1 L BMMY (1% yeast extract, 2% peptone, 0.23% potassium phosphate monobasic, 1.18% potassium phosphate dibasic, 1.34% yeast nitrogen base, 4×10⁻⁵ percent biotin and 0.5% methanol) to an OD₆₀₀ of 5, to induce expression, and grown at 30° C. with shaking at 300 rpm. Methanol was added to a final concentration of 2% every 24 h to maintain induction. Following five days of induction, the culture was centrifuged again, and the supernatant containing the secreted recombinant inhibitors was prepared for purification by nickel-immobilized metal affinity chromatography (IMAC).

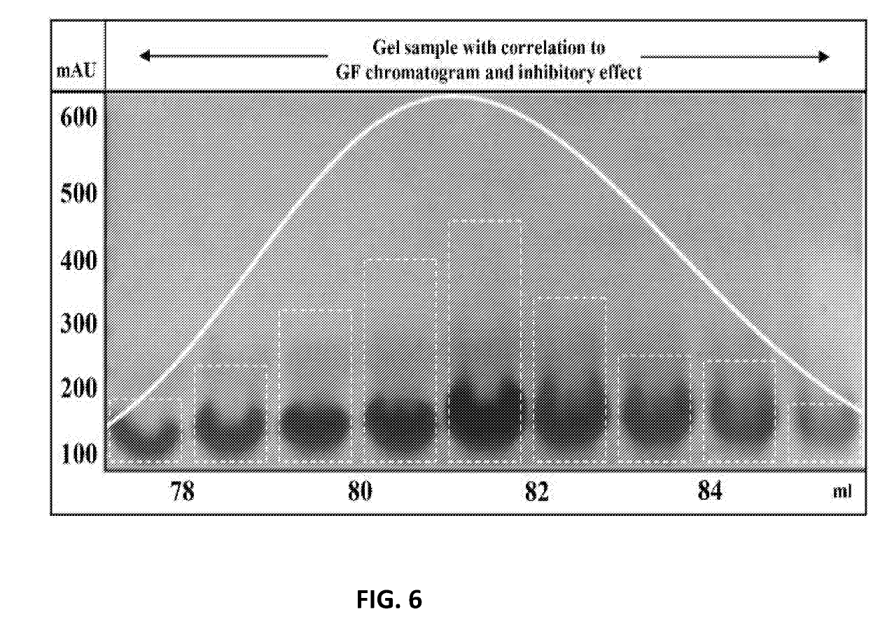

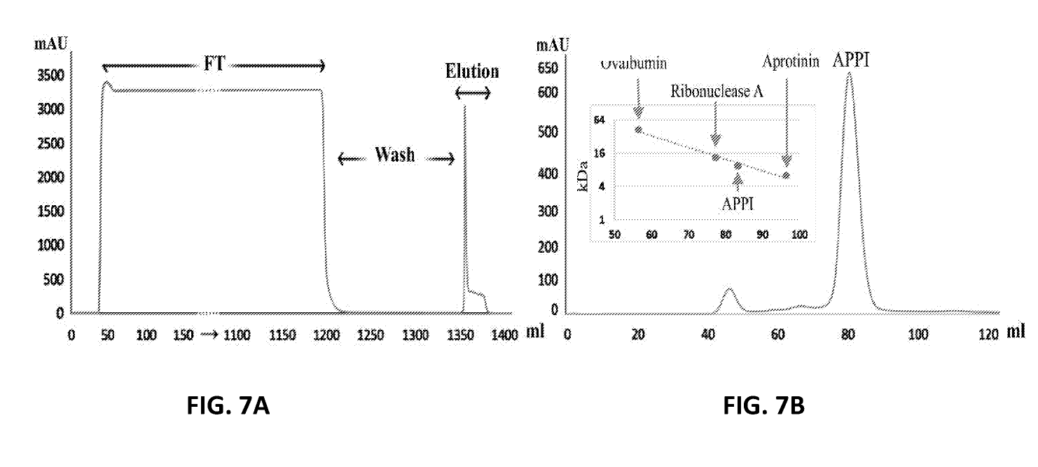

The supernatant containing the recombinant APPI was filtered through a Steritop bottle-top filter (Millipore). The filtered supernatant was adjusted to 10 mM imidazole and 0.5 M NaCl at pH 8.0 and left to stand overnight at 4° C. Thereafter, a second filtration was performed to remove any additional precipitation. The resulting supernatant was loaded on a HisTrap 5-ml column (GE Healthcare) at a flow rate of 0.7 ml/min for 24 h, washed with 20 mM sodium phosphate, 0.5 M NaCl, and 10 mM imidazole (pH 8.0) and eluted with 20 mM sodium phosphate, 0.5 M NaCl, 0.5 M imidazole (pH 8.0) in an ÄKTA pure instrument (FIG. 7A). The eluted inhibitors were concentrated, and the buffer was replaced with TB in a 3-kDa molecular weight cutoff (MWCO) Vivaspin concentrator (GE Healthcare). Gel filtration chromatography was performed using a Superdex 75 16/600 column (GE Healthcare) equilibrated with TB at a flow rate of 1 ml/min on an ÄKTA pure instrument (FIG. 7A-B). Gel filtration protein fractions were analyzed by SDS-PAGE on a 15% polyacrylamide gel under non-reducing conditions and tested for their ability to inhibit bovine trypsin catalytic activity (see experimental details below and FIG. 6). The correct mass of the pure proteins was validated using MALDI-TOF REFLEX-IV (Bruker), mass spectrometer.

Bovine Trypsin Activity Assay.

Assays were conducted at 37° C. in a Synergy2 plate reader spectrophotometer (BioTek). TB (185 μl), bovine trypsins (5 μl; 100 nM bovine trypsin), and APPI inhibitor (5 μl) were mixed and equilibrated prior to initiation of the reaction by the addition of the substrate, Z-GPR-pNA (5 μl; 1.5 mM). Reactions were followed spectroscopically for 5 min, and initial rates were determined from the increase in absorbance (410 nm) caused by the release of p-nitroaniline.

Far-UV Circular Dichroism Spectroscopy.

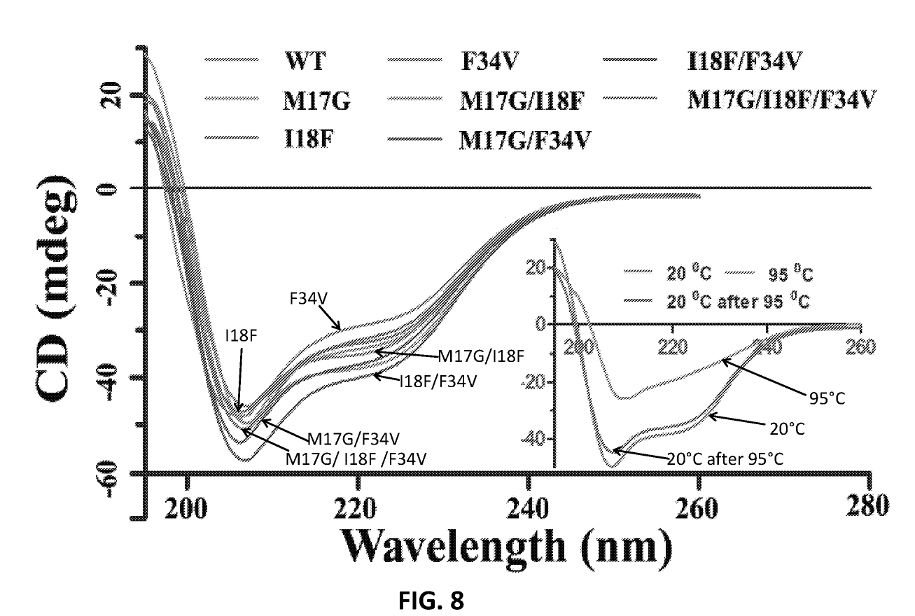

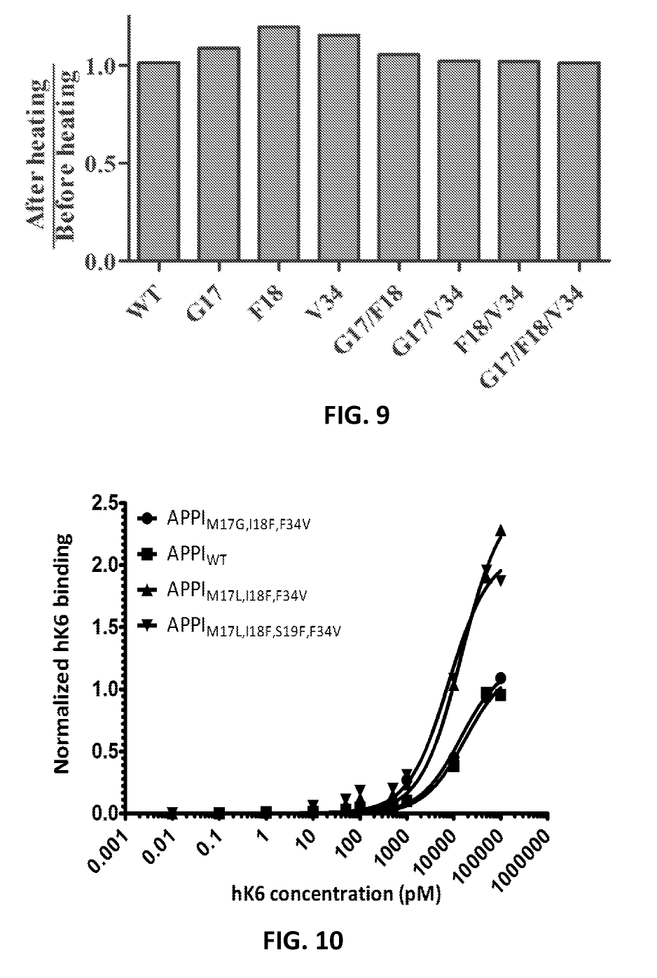

Circular dichroism (CD) spectra were recorded on a Jasco J-715 spectropolarimeter over a range of 190-260 nm using a quartz cuvette with a path length of 1 mm, a scanning speed of 50 nm/min and a data interval of 1 nm. Each sample of APPI variant was first analyzed at room temperature (20° C.), and left in the spectropolarimeter until 95° C. was reached (denaturation), then the sample was analyzed, cooled to 20° C. (renaturation), and analyzed again. Proteins were analyzed in TB. Three scans of 50 μM protein solutions were averaged to obtain smooth data and background corrected with respect to protein-free buffer (see FIG. 8).

### Example 1

### Yeast-Displayed APPI_{WT} is Rapidly Cleaved by Human Mesotrypsin

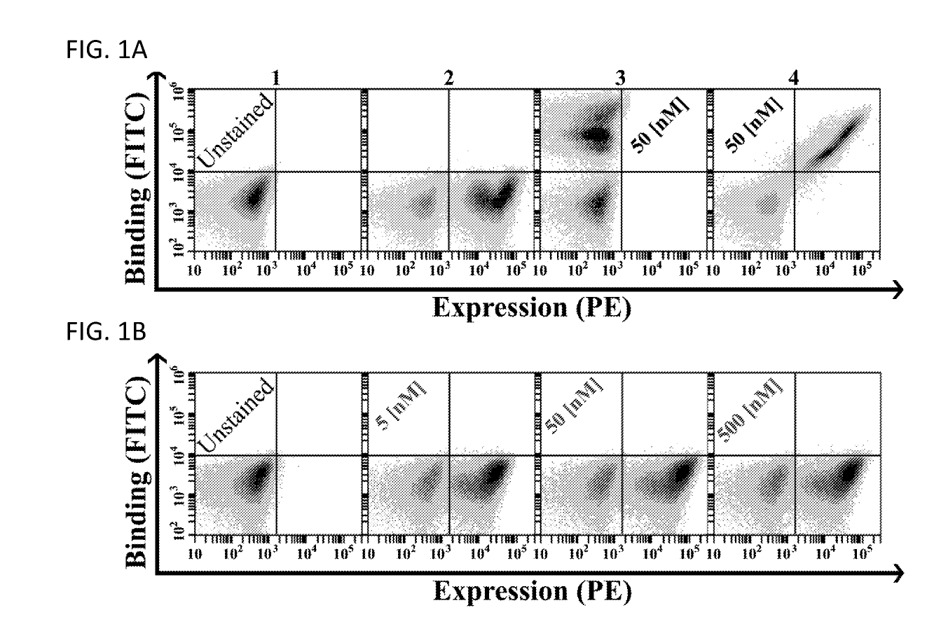

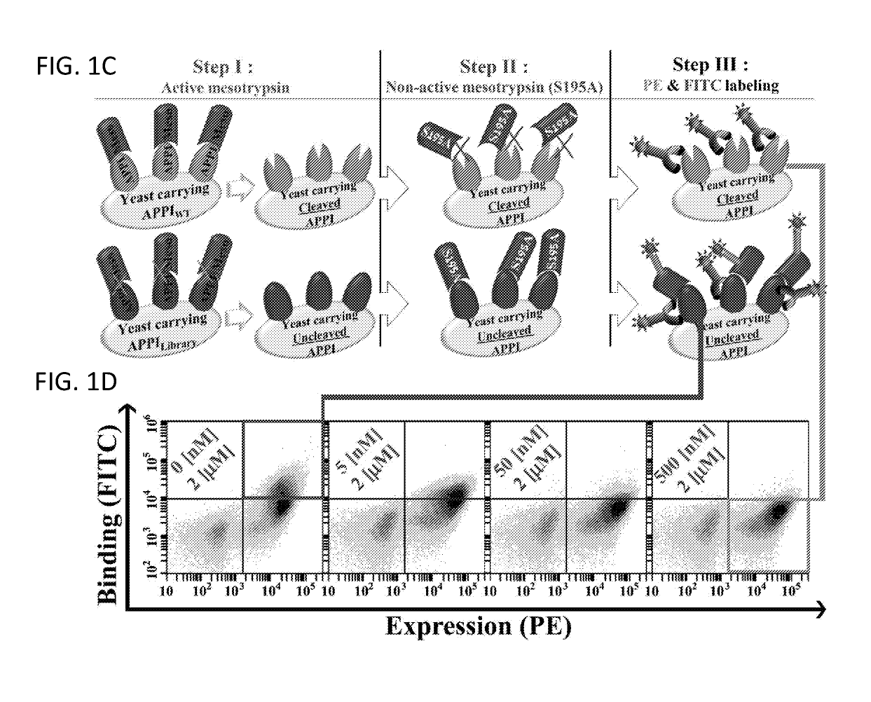

The yeast surface display (YSD) system for directed evolution is based on expression of a library of mutant proteins on the surface of yeast, followed by selection of variants with improved affinity. However, this system has not been employed previously for identifying proteolytic cleavage or improving the proteolytic resistance of a displayed inhibitor. To test the compatibility of APPI_{WT} with the YSD system, the coding region of APPI_{WT} was cloned into a YSD plasmid for presentation on the yeast S. cerevisiae surface as a fusion with the Aga2p agglutinin protein. Correct folding of APPI_{WT} was then verified using FACS by detection of bound fluorescently labeled bovine trypsin, which is an established, tight-binding target of APPI. As seen in FIG. 1A, APPI displayed on the yeast surface was highly expressed and showed significant binding to bovine trypsin, demonstrating proper folding of APPI (FIG. 1A).

Next, the ability of mesotrypsin to similarly detect APPI displayed on the yeast cell surface was assessed. Using a broad range of mesotrypsin concentrations, mesotrypsin binding was not detected (FIG. 1B). It was hypothesized that surface-displayed APPI may be rapidly proteolyzed by mesotrypsin, preventing detection of the transient binding event. This explanation would be consistent with the previously reported rapid cleavage of APPI by mesotrypsin in solution, and with the relatively long incubation time (at least 60 min) required for cell labeling prior to FACS. Challenged by the need to detect mesotrypsin binding uncoupled from proteolysis, a catalytically inactive form of mesotrypsin, in which the serine nucleophile is mutated to alanine (mesotrypsin-S195A) was employed. Unlike active mesotrypsin, mesotrypsin-S195A bound to surface-displayed APPI and resulted in a strong FACS signal (FIG. 1D, left panel). Additionally, it was found that preincubation of APPI-displaying yeast cells with active mesotrypsin prior to detection with mesotrypsin-S195A resulted in a concentration-dependent decrease in FACS signal (FIG. 1D, right panels), confirming the hypothesis that surface-displayed APPI is rapidly proteolyzed and depleted by mesotrypsin.

### Example 2

### Strategy for Proteolytic Stability Maturation of an APPI Library

Prompted by the discovery that active mesotrypsin proteolyzes surface-displayed APPI and that mesotrypsin-S195A can detect residual, uncleaved APPI on the cell surface, it was postulated that these reagents could be used in a stepwise fashion to enrich an APPI diversity library for variants with proteolytic resistance. As a starting point, a randomized library was generated in which mutations were introduced throughout the entire APPI gene at a frequency of 0-3 mutations per clone, producing a library of about 9×10⁶ independent variants. Diversity was introduced throughout the molecule, because while protease specificity is largely directed by the sequence of the canonical binding loop, it was previously found that proteolytic stability is a property strongly influenced by residues within the scaffold

The presented unique screening strategy, designated “triple staining”, was comprised of three steps (FIG. 1C). First, active mesotrypsin was incubated with the yeast displayed APPI library and allowed to cleave the less-resistant APPI clones. Second, active mesotrypsin was washed out and replaced with biotinylated mesotrypsin-S195A, which bound selectively to the uncleaved (resistant) clones. Third, the bound mesotrypsin-S195A was visualized by staining with fluorescently labeled streptavidin, facilitating detection. In directed evolution by yeast display (e.g., affinity maturation, or in general—“property” maturation), the sorting stringency is typically controlled by either the target concentration (equilibrium screening) or the dissociation time (kinetic screening). Because of the short enzymatic turnover time and comparatively long incubation time required for YSD APPI labeling for sorting by FACS, the reaction was let to reach steady state prior to sorting (i.e., 30 min of incubation with active mesotrypsin). Here, elevated concentrations of active mesotrypsin was used as an evolutionary stimulus, with the fluorescently labeled mesotrypsin-S195A as a marker to facilitate identification of the most proteolytically resistant APPI variants (see triple staining method, FIG. 2A).

Diagonal sorting gates were used for each of the sorts S3, S4 and S5 (where S stands for sort, and the number indicates the sort phase), which allow binding normalization versus expression in real-time during the flow-cytometric sorting process, thereby dramatically decreasing bias of the expression level (i.e., the avidity effect).

### Example 3

### High-Affinity and High-Stability Variants Identified at S5

‘Triple staining’ analysis of cells displaying APPI_{WT} and cells from the library maturation cycles (S1 to S5) showed that the more the sort is advanced, the stability and affinity for mesotrypsin is higher (FIG. 2B₁); remarkably is S5 that showed high tolerance to the proteolytic activity of mesotrypsin at all enzyme concentrations that were used. Having produced a library containing resistant clones, the inventors proceeded to determine whether it would be possible to detect the binding interaction between active mesotrypsin towards each of the stability maturation screening steps (as was done with bovine trypsin). Indeed, ‘double staining’ analysis of cells from sorts S1 to S5 with active mesotrypsin (without inactive mesotrypsin) showed high binding in the advanced sorting rounds (i.e., S4, S5, FIG. 2B₂). These results suggest a relatively high population of proteolytically resistant APPI variants in the S5 library (i.e., stability-matured variants).

### Example 4

### Identification of APPI Clones with Improved Resistance to Cleavage

DNA sequencing of 37 randomly selected APPI clones from S5 showed three repeating mutations, M17A, I18F, and F34V, along with a number of unique mutations (Table 1).

Staining of the YSD clones with active mesotrypsin showed that M17G and I18F exhibited high binding affinity and proteolytic stability, whereas F34V had only marginally enhanced binding affinity and stability vs. APPI_{WT} (FIG. 2C). The three mutations are spatially close to each other in the three-dimensional structure of APPI, and may be expected to interact physically. To better understand the potential functional interactions among mutations, the effect of all possible combinations was investigated (FIG. 2C), allowing assessment of additive, cooperative (beneficial dependence), or uncooperative (harmful dependence) interactions among mutations with respect to affinity and proteolytic resistance. Interestingly, the results imply an additive or cooperative effect, in which the triple mutant showed a remarkably higher binding affinity and proteolytic stability than the other combinations.

### Example 5

### Affinity/Stability-Matured APPI Variants Show Improvements in Mesotrypsin Inhibition

The YSD data shown in FIG. 2C reflect the net effect of mutations on proteolytic stability and mesotrypsin affinity, but do not distinguish between these two parameters. To accurately assess mesotrypsin affinity and proteolytic stability independently, soluble forms of the mutant proteins were expressed and purified. Inhibition constants (Kᵢ), approximating the enzyme-inhibitor dissociation constants (K_{d}), were determined by testing APPI_{WT} and mutated variants as inhibitors of mesotrypsin catalytic activity against the small chromogenic peptide substrate Z-GPR-pNA. A classic competitive pattern of inhibition for all inhibitors (FIG. 3A, 3B) was observed, measuring a Kᵢ value for APPI_{WT} of 131±17 nM (Table 2). APPI_{M17G} showed ˜40-fold improvement in Kᵢ and APPI_{I18F} showed a similar improvement, whereas APPI_{F34V} showed ˜3-fold improvement in mesotrypsin affinity (Table 2).

TABLE 2
Kinetic constants of mesotrypsin with APPI variants

| SEQ ID | | | | | Turnover | Turnover |
|---|---|---|---|---|---|---|
| NO | Inhibitor | Kᵢ (M) | Kᵢ (fold) | K_{cat} (s⁻¹) | time (s) | time (fold) |
| 25 | APPI-WT | *(1.31 ± 0.17) × 10⁻⁷ | 1 | (35.6 ± 2.3) × 10⁻³ | 28.1 ± 1.8 | 1 |
| 23 | APPI-F34V | *(5.01 ± 0.46) × 10⁻⁸ | 2.6 | (16.1 ± 1.6) × 10⁻³ | 62.4 ± 4.3 | 2.2 |
| 18 | APPI-M17G | **3.69 × 10⁻⁹ | 34.8 | | | |
| 18 | APPI-M17G | *(3.29 ± 0.25) × 10⁻⁹ | 39.8 | (15.6 ± 1.5) × 10⁻³ | 64.3 ± 6.4 | 2.3 |
| 19 | APPI-I18F | *(3.29 ± 0.21) × 10⁻⁹ | 39.8 | (10.4 ± 0.9) × 10⁻³ | 96.5 ± 8.2 | 3.4 |
| 20 | APPI-I18F/F34V | **(3.3 ± 0.07) × 10⁻⁹ | 39.8 | (5.35 ± 0.2) × 10⁻³ | 187.1 ± 7.7 | 6.6 |
| 21 | APPI-M17G/F34V | **(1.40 ± 0.11) × 10⁻⁹ | 93.6 | (37.1 ± 1.4) × 10⁻⁴ | 270.0 ± 10.1 | 9.6 |
| 22 | APPI-M17G/I18F | **(45.25 ± 0.36) × 10⁻¹¹ | 290 | (29.1 ± 0.6) × 10⁻⁴ | 344.0 ± 7.6 | 12.2 |
| 8 | APPI-M17G/I18F/ | **(89.8 ± 0.23) × 10⁻¹² | 1459 | (4.29 ± 0.3) × 10⁻⁴ | 2336.7 ± 140.0 | 83 |
| | F34V | | | | | |

* and ** represent fitted to Equations 1 and 2, respectively.
^{‡}Values are means ± SD

Considering that the lowest Kᵢ values of our single-mutation APPI variants are in the lower nano-molar range, close to the practical limit of 1-2 nM for Kᵢ determination using the classical competitive inhibition equation (Williams and Morrison, Methods Enzymol. 1979; 63:437-67), it was not possible to apply this method for combination variants expected to exhibit much lower Kᵢ values as a slow, tight binders (assuming additive or cooperative effect). Hence, the assumption of slow, tight binding behavior required a different kinetic treatment, as shown in FIG. 3C-3F and summarized in Table 2. In order to compare the results obtained from the slow-tight binding vs. the classical competitive inhibition studies, APPI_{M17G} inhibition was evaluated using both approaches, with results that showed a high correlation between the two methods (FIG. 3A-3D, Table 2). As anticipated, the Kᵢ values for double and triple mutants were for the most part significantly enhanced compared to the single mutants (Table 2). In particular, an outstanding improvement in binding—of more than three orders of magnitude—of the triple mutant variant (Kᵢ=89.8 μM) vs. the wild type (Kᵢ=131,000 μM) was observed (FIG. 3E, 3F and Table 2).

In addition to affinity and stability, inhibitor specificity is another significant factor for in-vivo applications. The S1 peptidase family to which mesotrypsin belongs is one of the largest protease families in the human degradome with over 100 enzymes, and contains ˜80 active proteases that, like mesotrypsin, have tryptic-like specificity for cleavage after Lys or Arg, and thus represent alternative targets for APPI and its variants. These enzymes therefore may acts as modulators of in-vivo APPI concentrations (i.e., mesotrypsin competitors), and their inhibition by may potentially lead to unwanted off-target effects of engineered mesotrypsin inhibitors. To test the specificity of the APPI_{M17G/I18F/F34V} triple mutant, cationic trypsin, anionic trypsin, Factor XIa (FXIa) and Kallikrein-6 (KLK6) were selected as targets that bind tightly to APPI_{WT} and therefore serve as competitors for in-vivo mesotrypsin binding. Importantly, it was found that while APPI_{M17G/I18F/F34V} shows greatly improved binding affinity toward mesotrypsin by comparison with APPI_{WT}, affinity improvements toward KLK6, cationic and anionic trypsins are negligible, and affinity is substantially weakened toward APPI physiological target FXIa. Thus, the mutations present in APPI_{M17G/I18F/F34V} (SEQ ID NO: 8) result in enhancement of specificity toward mesotrypsin over other proteases by three to five orders of magnitude (Table 3).

TABLE 3
The inhibitor specificity of APPI-M17G/ I18F/ F34V towards a range of human serine proteases

| | Kᵢ for meso- | Kᵢ for kallikrein-6 | Kᵢ for cationic | Kᵢ for anionic | |
|---|---|---|---|---|---|
| Inhibitor | trypsin (M) | (M) | trypsin (M) | trypsin (M) | Kᵢ for FXIa (M) |
| APPI-WT (SEQ ID NO: 25) | (1.31 ± 0.17) × 10⁻⁷ | (2.23 ± 0.18) × 10⁻⁹ | (6.27 ± 1.01) × 10⁻¹² | (1.74 ± 0.05) × 10⁻¹² | (4.1 ± 0.14) × 10⁻¹⁰ |
| APPI-M17G/ I18F/ F34V | (89.8 ± 0.23) × 10⁻¹² | (1.09 ± 0.12) × 10⁻⁹ | (4.96 ± 0.25) × 10⁻¹² | (1.47 ± 0.02) × 10⁻¹² | (9.84 ± 0.32) × 10⁻⁸ |
| (SEQ ID NO: 8) | | | | | |
| Kᵢ (fold) | 1459 | 2.04 | 1.3 | 1.18 | 4.16 × 10⁻³ |
| $\begin{matrix}Specificity \\ ( \frac{{K}_{i} ( fold ) for X}{{K}_{i} ( fold ) for Mesotrypsin} )\end{matrix}$ | 1 | 715 | 1122 | 1236 | 350000 |

Example 5 indicates that APPI_{M17G/I18F/F34V} is a suitable candidate for in-vivo applications targeting mesotrypsin.

### Example 6

### Triple Mutant Cycle Analysis of the Interactions Between Residues at Positions 17, 18 and 34 in APPI

For the APPI-mesotrypsin complex, it was found that each of the mutations impacted the strength of binding and the rate of hydrolysis to different degrees. The data generated in this study enabled us to assess the extent to which the effects of the mutations on the measured functional properties (Kᵢ and k_{cat}) are independent (non-cooperative) or cooperative. The strength of the (direct or indirect) interactions between residues X and Y in a protein (P) can be determined by constructing a cycle that comprises the wild-type protein P_{XY}, two single mutants, P_{X0} and P_{0Y}, and the corresponding double mutant, P₀₀ (0 indicates a mutation). A measure for the strength of interaction is the coupling energy, ΔΔGᵢₙₜ, which is given by:

$$ΔΔ {G}_{int} = Δ G ( {P}_{XY} ) - Δ G ( {P}_{0 Y} ) - Δ G ( {P}_{X 0} ) + G ( {P}_{00} ) = - RT ln ( \frac{{K}_{XY} \times {K}_{00}}{{K}_{0 Y} \times {K}_{X 0}} ) \qquad Equation 3$$

where R is the gas constant, T is the absolute temperature and ΔG(P_{XY}), ΔG(P_{0Y}), ΔG(P_{X0}) and ΔG(P₀₀) correspond to the free energies of binding or catalysis. A coupling energy of zero (i.e. additivity of mutational effects) indicates that there is no interaction between X and Y with respect to the process (e.g. association) that is considered.

The free energy changes of catalysis (ΔΔG_{cat}) and association (ΔΔGₐ) upon single point mutation (e.g. ΔΔG of P_{XY} and P_{X0}) were calculated in a similar manner using Eq. 4:

$$ΔΔ G = Δ G ( {P}_{X 0} ) - Δ G ( {P}_{XY} ) - RT ln \frac{{K}_{X 0}}{{K}_{XY}} \qquad Equation 4$$

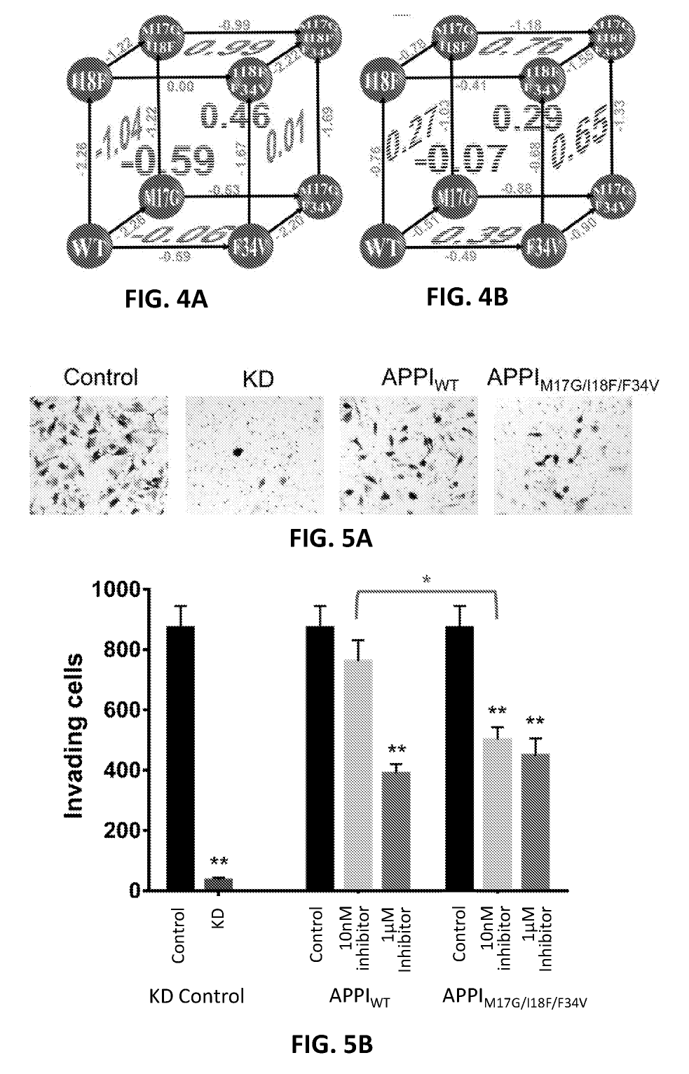

Previous studies of free energy of coupling have suggested an energy values with errors (from zero) of X kcal/mol in order to assume additivity. Indeed, our free energy of coupling results for catalysis and association (ranging from −1.04 to 0.99 kcal/mol) suggest that each mutation is independent (FIG. 4), therefore energetically additive. Because the changes in free energy for each cycle are calculated using experimentally measured k_{cat} and Kᵢ values from eight different variants (with the associated experimental errors), small non-zero values for the free energy of coupling may be explained by experimental error.

### Example 7

### APPI_{M17G/I18F/F34V} Variant Reveals Enhanced Potency for Inhibition of Mesotrypsin-Dependent Cancer Cell Invasiveness

Previously, mesotrypsin was implicated as an enzyme responsible for mediating invasiveness and malignant morphology of prostate cancer cells. To evaluate the ability of the APPI_{M17G/I18F/F34V} triple mutant (SEQ ID NO: 8) to inhibit these phenotypes, experiments were carried out using human PC3-M prostate cancer cells, a hormone-independent, highly aggressive and metastatic cell line (Kozlowski, J. M., et al., Cancer research, 1984. 44(8): p. 3522-9). In Matrigel transwell invasion assays, it was confirmed that mesotrypsin expression is essential for the invasiveness of these cells, since transduction with a lentiviral shRNA construct targeting the PRSS3 gene (encoding mesotrypsin) results in profound inhibition of invasiveness (FIG. 5A, B, KD control), as previously reported (Hockla, A., et al., Mol Cancer Res, 2012. 10(12): p. 1555-66.). When control cells with endogenous PRSS3 expression were treated with 10 nM APPI_{M17G/I18F/F34V}, significant inhibition of invasion was observed by comparison with control cells, whereas 10 nM APPI_{WT} did not produce a significant effect. At much higher inhibitor concentrations (1 μM), both inhibitors produced similar maximum inhibitory effects of ˜50%. This experiment demonstrates enhanced potency of APPI_{M17G/I18F/F34V} compared with APPI_{WT} for suppression of cellular invasiveness. The inability of either inhibitor to suppress invasion to the same extent as mesotrypsin knockdown may result from inadequate selectivity, as a result of competition for binding from other proteases in the cellular milieu; this possibility suggests the value of continued engineering efforts to further enhance the selectivity of mesotrypsin-targeted APPI variants.

### Example 8

### Additional Effective APPI Variant

Using a Pichia pastoris expression system, additional soluble APPI variants bearing several mutations were produced. Mutations in APPI_{T11V/M17G/I18F/F34V} and APPI_{M17G/I18F/K29L/F34V} contributed to striking improvements in affinity and proteolytic resistance relative to WT-APPI (Table 4). The mutants APPI_{T11V/M17G/I18F/F34V} and APPI_{M17C/I18F/F34C}, showed around 2000-fold improvement in affinity and around 100-fold improvement in proteolytic stability relative to APPI-WT. The mutant APPI_{M17G/I18F/K29L/F34V}, showed around 1000-fold improvement in affinity and around 60-fold improvement in proteolytic stability relative to APPI-WT. The mutant APPI_{T11C/M17G/I18F/F34C}, showed similar affinity and around 2-fold improvement in proteolytic stability relative to APPI-WT.

TABLE 4
Affinity and proteolytic resistance of additional APPI variants

| | | | | | turnover | turnover |
|---|---|---|---|---|---|---|
| | Ki (pM) | Ki SD (pM) | Kcat (1/s) | Kcat SD (1/s) | time (s) | time SD (s) |
| APPI_{T11V/M17G/I18F/F34V} | 49.88 | 0.78 | 0.000347 | 1.69E−05 | 2880.96 | 136.66 |
| (SEQ ID NO: 9) | | | | | | |
| APPI_{M17G/I18F/K29L/F34V} | 89.06 | 0.95 | 0.000535 | 1.68E−05 | 1868.76 | 59.66 |
| (SEQ ID NO: 10) | | | | | | |
| APPI_{T11C/M17G/I18F/F34C} | 148800 | 10630 | 0.00521 | 0.00021 | 192.21 | 7.69 |
| (SEQ ID NO: 11) | | | | | | |
| APPI_{M17C/I18F/F34C} | 52.4 | 0.78 | 0.00038 | | 2631 | |
| (SEQ ID NO: 12) | | | | | | |

### Example 9

### APPI Variants as a ⁶⁴Cu-Radiolabeled Tumor-Targeted Imaging Agent

Derivatization and Radiolabeling of the Best APPI-Derived Mesotrypsin Inhibitor and Evaluation of In Vivo Stability, Pharmacokinetics, Tumor Uptake and Biodistribution Profile.

A cross-bridged DOTA (1,4,7,10-tetraazacyclododecane-1,4,7,10-tetraacetic acid) chelator is used to derivatize the best APPI variant as a chemical handle for subsequent ⁶⁴Cu-radiolabeling to enable in vivo pharmacokinetic and imaging studies. For site-specific conjugation without disruption of mesotrypsin binding affinity or biological activity, a mutant with substitution of the one native lysine residue is used. DOTA chelator-modified protein (20 μg) is radiolabeled using ⁶⁴CuCl₂ (100-500 MBq) after optimization of pH values, reaction temperatures and times. Radiolabeled protein is purified using a SPE (solid phase extraction) protocol followed by sterile filtration. The radiochemical and chemical purity of the purified product (295% required) is determined by analytical radio-HPLC.

Stability and Pharmacokinetics of the ⁶⁴Cu-Labeled APPI Protein.

The ⁶⁴Cu-labeled APPI variant is incubated in serum for 0.5, 1, 4, and 24 h at 37° C. Thereafter, the serum proteins may be precipitated using acetonitrile, and the solution is analyzed by radio-HPLC for the presence of intact peptide, fragments, or free ⁶⁴Cu. In addition, in vivo studies are carried out. For this purpose, 3 anesthetized male Nod/Scid mice for each radiotracer and time-point are injected into the lateral tail vein with 5-10 MBq of the ⁶⁴Cu-labeled ligands. The animals are sacrificed at 0.5, 1, 2, 4, and 8 h post injection. Blood, kidney and liver are removed, the relevant tissues will be homogenized, and the homogenates are extensively filtered using the Nanosep 10K Omega filter (Pall Corporation). The filtrates are analyzed by radio-HPLC for the presence of intact peptides, fragments, or free ⁶⁴Cu.

In Vivo Evaluation of the ⁶⁴Cu-Labeled Proteins.

Initial studies employ the orthotopic PC3-M model of human prostate cancer. Subsequent studies implement additional orthotopic models of pancreatic, breast and lung cancers.

μPET Imaging.

Anesthetized mice bearing PC-3M tumors are injected (via the lateral tail vein) with 5-10 MBq of ⁶⁴Cu-labeled protein and imaged on a μPET/CT system. Images are acquired after 0.5, 1, 2, 4, and 24 h (15 min for each scan). To test mesotrypsin binding specificity in vivo, a control group is co-injected with an excess of unlabeled APPI protein.

Biodistribution Studies.

Mice are euthanized following intravenous administration of the radiolabeled APPI variant at the optimal time-point determined above. Blood, heart, lung, liver, spleen, pancreas, stomach, intestine, skin, muscle, bone, brain, tail, and tumor tissue are removed, and the radioactivity in each organ is determined by γ-counting. Results are expressed as the % ID/g of tissue. For each mouse, the activity of tissue samples is calibrated against a known aliquot of the radiotracer and is normalized to the whole body weight and to the residual activity present in the tail.

Post-Imaging Tumor Analysis.

Tumors are excised after in vivo imaging and sectioned using a cryostat (Leica Microsystems, Bannockburn, Ill.). Tumor sections are analyzed with high resolution autoradiography using a PhosphorImager SI (Amersham Biosciences, Piscataway, N.J.). Adjacent tumor sections are analyzed by immunohistochemistry, using appropriate antibodies to visualize expression of mesotrypsin and the results of immunohistochemistry and autoradiography is correlated.

### Example 10

### Generation of APPI Variants that Serve as High Affinity hK6 Inhibitors

### Generation of a Combinatorial APPI Based Library

A library of variants of APPI_{M17G,I18F,F34V} (SEQ ID NO: 8) which exhibits great stability and resistance toward cleavage by human mesotrypsin, was generated. The library of variant of SEQ ID NO: 8 was screened to isolate high affinity hK6 inhibitors. The APPI_{M17G,I18F,F34V} gene was constructed by using codons optimized for yeast expression and synthesized by PCR-assembly. The APPI library was constructed using PCR-based NNS randomization strategy and error-prone PCR, generating a library with 1-2 mutations per clone, including one loop mutation (positions T11-F18) that is essential for binding to hK6.

### Flow Cytometry and Cell Sorting

Screening of the library was performed using a yeast surface display (YSD) system. Five different high affinity clones were identified.

| | |
|---|---|
| APPI_{M17L,I18F,S19F,F34V} | EVCSEQAETGPCRALFFRWYFDVTEGKCAPFVYGGCGGNRNNF |
| (SEQ ID NO: 13) | DTEEYCMAVCGSAI |
| APPI_{M17L,I18F,F34V} | EVCSEQAETGPCRALFSRWYFDVTEGKCAPFVYGGCGGNRNNF |
| (SEQ ID NO: 14) | DTEEYCMAVCGSAI |
| APPI_{M17H,I18F,F34V} | EVCSEQAETGPCRAHFSRWYFDVTEGKCAPFVYGGCGGNRNNF |
| (SEQ ID NO: 15) | DTEEYCMAVCGSAI |
| APPI_{M17S,I18F,F34V} | EVCSEQAETGPCRASFSRWYFDVTEGKCAPFVYGGCGGNRNNFD |
| (SEQ ID NO: 16) | TEEYCMAVCGSAI |
| APPI_{M17F,I18F,F34V} | EVCSEQAETGPCRAFFSRWYFDVTEGKCAPFVYGGCGGNRNNFD |
| (SEQ ID NO: 17) | TEEYCMAVCGSAI |

### Production and Purification of APPI Proteins.

APPI WT (SEQ ID NO: 25), and polypeptides having the amino acid sequence of SEQ ID NO: 8, 13, and 14 were expressed in Pichia pastoris yeast strain together with an His tag at the C-terminal. All variants were purified using affinity chromatography (with nickel columns) and later using SEC (with superdex 75 column).

### Evaluation of the Clones Using YSD

Clones were isolated and expressed in the YSD system and their ability to bind human Kallikrein-6 was evaluated. Two high affinity clones were identified and selected namely, polypeptides having an amino acid sequence as set forth in SEQ ID NO: 13 and SEQ ID NO: 14.

The ability of the APPI variant having SEQ ID NO: 13 to inhibit mesotrypsin activity was confirmed, and the resulting Ki value was 5.38 nM±0.28 nM.

A titration curve was generated in order to estimate the K_{D} differences between APPI WT (SEQ ID NO: 25), APPI_{M17G,I18F,F34V} (SEQ ID NO: 8), APPI_{M17L,I18F,F34V} (SEQ ID NO: 14) and APPI_{M17L,I18F,S19F,F34V} (SEQ ID NO: 13). The resultant titration curve showed apparent K_{D} of 17.2 nM for APPI WT (SEQ ID NO: 25), 14.0 nM for SEQ ID NO: 8, 14.6 for SEQ ID NO: 14, and 7.8 nM for SEQ ID NO: 13 (FIG. 10).

Next, different concentrations of the polypeptides having SEQ ID Nos: 8, 13, and 14 were tested for their ability to bind 50 nM hK6 in the presence of a small molecule which target and home the serine residue in the active pocket of hK6.

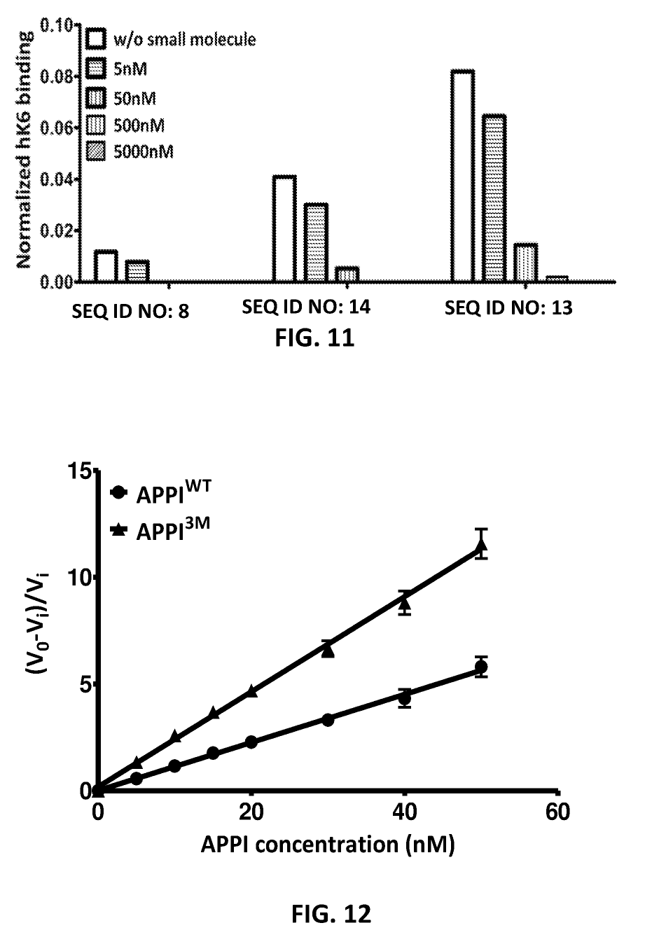

Results demonstrate higher binding of peptides having SEQ ID Nos: 13 and 14 to hK6 compared with the protein of SEQ ID NO: 8 (FIG. 11). Results further demonstrate that the binding of the two variants having SEQ ID Nos: 13 and 14 was diminished in the presence of high concentrations of the small molecule (FIG. 11).

Evaluation of the Clones in their Soluble Form

APPI WT (SEQ ID NO: 25) and APPI_{M17G,I18F,F34V} (SEQ ID NO: 8), were tested for their ability to inhibit hK6 catalytic activity. The inhibition constants (Ki) for APPI variants were calculated assuming slow tight inhibition mechanism.

Results demonstrated that polypeptides having SEQ ID NO: 25 (APPI^{WT}) and SEQ ID NO: 8 (APPI^{3M}) inhibited the catalytic activity of hK6 in low nano-molar range with Kᵢ values of 2.24 nM and Ki=1.1 nM, respectively, assuming slow tight inhibition mechanism (FIG. 12).

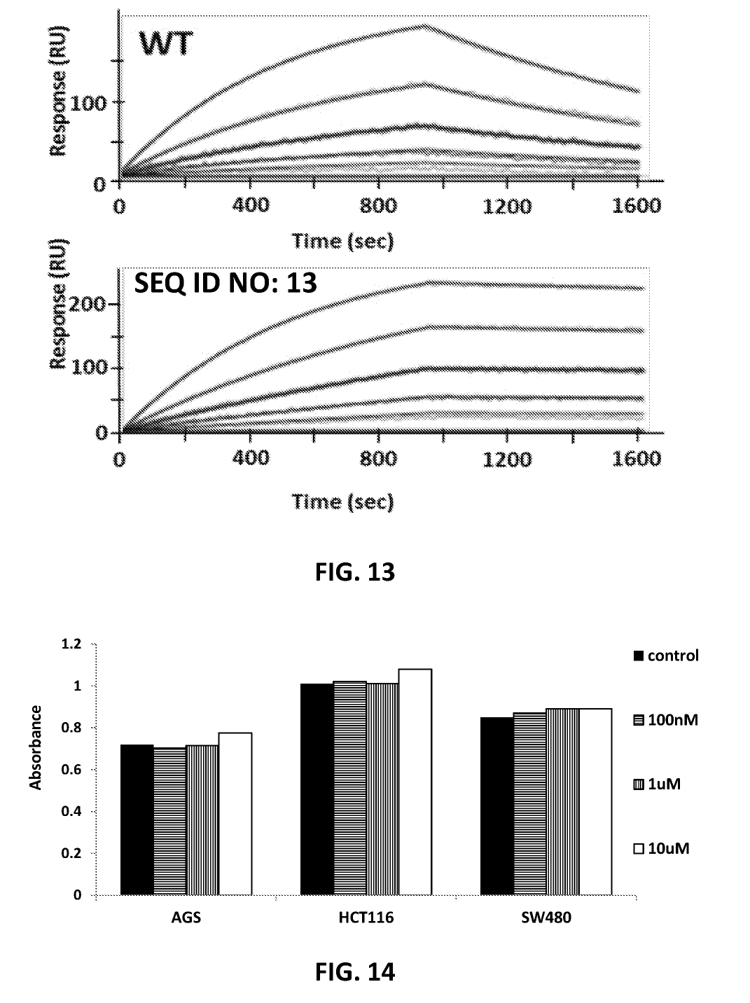

Further, APPI WT (SEQ ID NO: 25) and the variant having the amino acid sequence of SEQ ID NO: 13 were also evaluated for their ability to bind hK6 on a Surface Plasmon Resonance system. To this end, APPIs were mounted on a surface Plasmon resonance (SPR) nickel chip by their His tag, and hK6 molecules served as the analyte. SPR results showed 22.5 folds improvement in binding to hK6 for the polypeptide of SEQ ID NO: 13 (K_{D}=351 μM) compared to APPI WT (K_{D}=7.91 nM) (FIG. 13).

### Example 11

### Effect of APPI Variant on Proliferation and Invasion of Cancer Cells

The effect of an APPI variant (SEQ ID NO: 13) on proliferation of gastric cancer cells was evaluated by XTT assay. AGS, HCT-116 and SW-480 cells were plated into 96-well plates in 9600 cells/well in duplicate, and allowed to adhere for 4 hours. Each well was supplemented with 100 nM, 1 μM, or 10 μM APPI_{M17L,I18F,S19F,F34V} (SEQ ID NO: 13) or a vehicle (50 mM Tris-HCl, 100 mM NaCl, Ph 7.3), and cells were incubated for 48 hours. Proliferation was measured by XTT following the manufacturer instructions (Biological Industries, Ill.).

As shown in FIG. 14, the APPI variant (SEQ ID NO: 13) did not inhibit proliferation in all 3 cell lines, in all concentrations.

The effects of APPI_{M17L,I18F,S19F,F34V} (SEQ ID NO: 13) on the invasive behavior of AGS gastric cancer cells was examined. For invasion assays AGS cells were plated in the top chamber of a Matrigel coated ThinCerts (Greiner Bio-One, Germany), with an 8 μm pored membrane in 160 μl serum-free Ham's F-12 medium, in triplicate. 1 uM-10 uM of APPI (SEQ ID NO: 13) or a vehicle (50 mM Tris-HCl, 100 mM NaCl, Ph 7.3) in 40 μl were added to each insert. Next, the inserts were placed into the bottom chamber wells of a 24-well plate containing Ham's F-12 with 10% FBS as a chemo-attractant. After 48 hours of incubation, cells remaining on the inserts' top layers were removed by cotton swab scrubbing; Cells on the lower surface of the membrane were fixed in 100%/methanol and stained with Romanowski stain solutions. The cell numbers in 10 random fields (×20) were counted for each chamber and the average value was calculated.

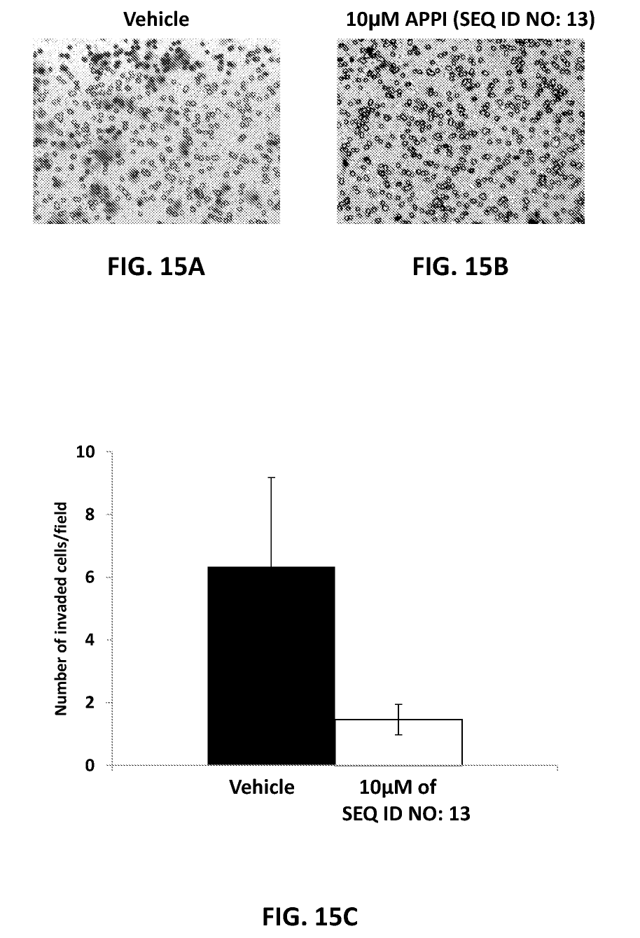

As shown in FIG. 15A-C, AGS cells treated expressing APPI variant (SEQ ID NO: 13) displayed significantly lower transmembrane invasion capacity compared with those treated with vehicle. The invasion capacity was reduced by 77%/a, with 6.33±2.85 invasive cells/field in control and 1.46±0.48 cells/field in APPI 10M treated cells.

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

What is claimed is:

1. An isolated polypeptide comprising the amino acid of SEQ ID NO: 1
(EVCSEQAEX₁GPCRAX₂X₃X₄RWYFDVTEGX₅CAPFX₆YGGCGGNRNNFDTEEYCMAVCG SAI) wherein:
X₁ is threonine, serine, cysteine or valine;
X₂ is glycine, cysteine, leucine, histidine, serine, phenylalanine or alanine;
X₃ is phenylalanine, leucine, tyrosine or tryptophan;
X₄ is serine or phenylalanine;
X₅ is lysine, isoleucine, leucine or methionine; and
X₆ is valine, cysteine, isoleucine, leucine or methionine;
or a fragment, a derivative or analog thereof.

2. The isolated polypeptide of claim 1, wherein X₁ is threonine, serine, cysteine or valine; X₂ is glycine, cysteine or alanine; X₃ is phenylalanine, leucine, tyrosine or tryptophan; X₄ is serine; X₅ is lysine, isoleucine, leucine or methionine; and X₆ is valine, cysteine, isoleucine, leucine or methionine, or a fragment, a derivative or analog thereof.

3. The isolated polypeptide of claim 1, wherein X₁ is threonin or valine; X₂ is glycine; X₃ is phenylalanine; X₄ is serine; X₅ is lysine or leucine; X₆ is valine, or a fragment, a derivative or analog thereof.

4. The isolated polypeptide of claim 1, wherein X₁ is cysteine, valine or threonine; X₂ is glycine or cysteine; X₃ is phenylalanine; X₄ is serine; X₅ is lysine or leucine; and X₆ is cysteine, or a fragment, a derivative or analog thereof.

5. The isolated polypeptide of claim 1, wherein X₁ is threonine; X₂ is glycine, leucine, histidine, serine or phenylalanine; X₃ is phenylalanine; X₄ is serine or phenylalanine; X₅ is lysine; and X₆ is valine, or a fragment, a derivative or analog thereof.

6. The isolated polypeptide of claim 1, wherein X₁ is threonine; X₂ is glycine or leucine; X₃ is phenylalanine; X₄ is serine or phenylalanine; X₅ is lysine; and X₆ is valine, or a fragment, a derivative or analog thereof.

7. The isolated polypeptide of claim 1, wherein X₁ is threonine; X₂ is leucine; X₃ is phenylalanine; X₄ is serine or phenylalanine; X₅ is lysine; and X₆ is valine, or a fragment, a derivative or analog thereof.

8. The isolated polypeptide of claim 1, comprising the amino acid sequence selected from the group consisting of SEQ ID NO: 8-14 or a fragment, a derivative or analog thereof.

9. The isolated polypeptide of claim 1, comprising the amino acid sequence as set forth in SEQ ID NO: 8 (EVCSEOAETGPCRAGFSRWYFDVTEGKCAPFVYGGCGGNRNNFDTEEYCMAVCGSAI) or a fragment, a derivative or analog thereof.

10-14. (canceled)

15. The isolated polypeptide of claim 1 having a length of at most 80 amino acid residues.

16. The isolated polypeptide of claim 1, wherein said analog has at least 95% sequence identity to any one of SEQ ID NOs: 1-14, and wherein said analog differs by at least one amino acid residue compared to SEQ ID NO: 25.

17. A pharmaceutical composition comprising the polypeptide of claim 1 and a pharmaceutical acceptable carrier.

18. A method for treating cancer in a subject in need thereof, the method comprising the step of administering to said subject a pharmaceutical composition comprising an effective amount of an amino acid molecule comprising the amino acid selected from the group consisting of SEQ ID NOs: 1-14, and a pharmaceutical acceptable carrier, thereby treating cancer in a subject in need thereof.

19. The method of claim 18, wherein said cancer is a metastatic-associated cancer.

20. The method of claim 18, wherein said cancer is a mesotrypsin-associated cancer.

21. The method of claim 18, wherein said cancer is selected from the group consisting of prostate, lung, colon, breast, pancreas, gastric, non-small cell lung cancer (NSCLC) and metastasis thereof.

22. The method of claim 18, wherein said cancer is prostate cancer.

23. The method of claim 18, wherein said cancer is gastric cancer.

24. The method of claim 18, wherein said treating is inhibiting invasiveness of a cancerous cell.

25. A method for imaging a mesotrypsin associated and/or kallikrein-6 associated neoplastic tissue in a subject in need thereof, the method comprising the steps of:
administering an imaging reagent compound comprising: an effective amount of an amino acid molecule comprising the amino acid selected from the group consisting of SEQ ID NOs: 1-14, and an imaging agent to a subject, wherein said imaging reagent compound distributes in vivo; and
detecting the compound in said subject,
thereby imaging mesotrypsin associated and/or kallikrein-6 associated neoplastic tissue.

26-27. (canceled)


SEQUENCE LISTING (40 sequences)

SEQ ID NO: 1 - protein, 57 aa, Artificial sequence
  synthetic sequence
  MISC_FEATURE (9)..(9): X is threonine, serine, cysteine or valine
  MISC_FEATURE (15)..(15): X is glycine, cysteine, leucine, histidine, serine, phenylalanine or alanine
  MISC_FEATURE (16)..(16): X is phenylalanine, leucine, tyrosine or tryptophan
  MISC_FEATURE (17)..(17): X is serine or phenylalanine
  MISC_FEATURE (27)..(27): X is lysine, isoleucine, leucine or methionine
  MISC_FEATURE (32)..(32): X is valine, cysteine, isoleucine, leucine or methionine
EVCSEQAEXGPCRAXXXRWYFDVTEGXCAPFXYGGCGGNRNNFDTEEYCMAVCGSAI

SEQ ID NO: 2 - protein, 57 aa, Artificial sequence; identical to SEQ ID NO: 1
  Synthetic sequence
  MISC_FEATURE (9)..(9): X is threonine, serine, cysteine or valine
  MISC_FEATURE (15)..(15): X is glycine, cysteine or alanine
  MISC_FEATURE (16)..(16): X is phenylalanine, leucine, tyrosine or tryptophan
  MISC_FEATURE (17)..(17): X is serine
  MISC_FEATURE (27)..(27): X is lysine, isoleucine, leucine or methionine
  MISC_FEATURE (32)..(32): X is valine, cysteine, isoleucine, leucine or methionine

SEQ ID NO: 3 - protein, 57 aa, Artificial sequence; identical to SEQ ID NO: 1
  Synthetic sequence
  MISC_FEATURE (9)..(9): X is threonine or valine
  MISC_FEATURE (15)..(15): X is glycine
  MISC_FEATURE (16)..(16): X is phenylalanine
  MISC_FEATURE (17)..(17): X is serine
  MISC_FEATURE (27)..(27): X is lysine or leucine
  MISC_FEATURE (32)..(32): X is valine

SEQ ID NO: 4 - protein, 57 aa, Artificial sequence; identical to SEQ ID NO: 1
  Synthetic sequence
  MISC_FEATURE (9)..(9): X is threonine
  MISC_FEATURE (15)..(15): X is glycine, leucine, histidine, serine or phenylalanine
  MISC_FEATURE (16)..(16): X is phenylalanine
  MISC_FEATURE (17)..(17): X is serine or phenylalanine
  MISC_FEATURE (27)..(27): X is lysine
  MISC_FEATURE (32)..(32): X is valine

SEQ ID NO: 5 - protein, 57 aa, Artificial sequence; identical to SEQ ID NO: 1
  Synthetic sequence
  MISC_FEATURE (9)..(9): X is threonine
  MISC_FEATURE (15)..(15): X is glycine or leucine
  MISC_FEATURE (16)..(16): X is phenylalanine
  MISC_FEATURE (17)..(17): X is serine or phenylalanine
  MISC_FEATURE (27)..(27): X is lysine
  MISC_FEATURE (32)..(32): X is valine

SEQ ID NO: 6 - protein, 57 aa, Artificial sequence; identical to SEQ ID NO: 1
  Synthetic sequence
  MISC_FEATURE (9)..(9): X is threonine
  MISC_FEATURE (15)..(15): X is leucine
  MISC_FEATURE (16)..(16): X is phenylalanine
  MISC_FEATURE (17)..(17): X is serine or phenylalanine
  MISC_FEATURE (27)..(27): X is lysine
  MISC_FEATURE (32)..(32): X is valine

SEQ ID NO: 7 - protein, 57 aa, Artificial sequence
  Synthetic sequence
EVCSEQAETGPCRAGFSRWYFDVTEGKCAPFVYGGCGGNRNNFDTEEYCMAVCGSAI

SEQ ID NO: 8 - protein, 57 aa, Artificial sequence
  Synthetic sequence
EVCSEQAEVGPCRAGFSRWYFDVTEGKCAPFVYGGCGGNRNNFDTEEYCMAVCGSAI

SEQ ID NO: 9 - protein, 57 aa, Artificial sequence
  Synthetic sequence
EVCSEQAETGPCRAGFSRWYFDVTEGLCAPFVYGGCGGNRNNFDTEEYCMAVCGSAI

SEQ ID NO: 10 - protein, 57 aa, Artificial sequence
  Synthetic sequence
EVCSEQAECGPCRAGFSRWYFDVTEGKCAPFCYGGCGGNRNNFDTEEYCMAVCGSAI

SEQ ID NO: 11 - protein, 57 aa, Artificial sequence
  Synthetic sequence
EVCSEQAETGPCRACFSRWYFDVTEGKCAPFCYGGCGGNRNNFDTEEYCMAVCGSAI

SEQ ID NO: 12 - protein, 57 aa, Artificial sequence
  Synthetic sequence
EVCSEQAETGPCRALFFRWYFDVTEGKCAPFVYGGCGGNRNNFDTEEYCMAVCGSAI

SEQ ID NO: 13 - protein, 57 aa, Artificial sequence
  Synthetic sequence
EVCSEQAETGPCRALFSRWYFDVTEGKCAPFVYGGCGGNRNNFDTEEYCMAVCGSAI

SEQ ID NO: 14 - protein, 57 aa, Artificial sequence
  Synthetic sequence
EVCSEQAETGPCRAHFSRWYFDVTEGKCAPFVYGGCGGNRNNFDTEEYCMAVCGSAI

SEQ ID NO: 15 - protein, 57 aa, Artificial sequence
  Synthetic sequence
EVCSEQAETGPCRASFSRWYFDVTEGKCAPFVYGGCGGNRNNFDTEEYCMAVCGSAI

SEQ ID NO: 16 - protein, 57 aa, Artificial sequence
  Synthetic sequence
EVCSEQAETGPCRAFFSRWYFDVTEGKCAPFVYGGCGGNRNNFDTEEYCMAVCGSAI

SEQ ID NO: 17 - protein, 57 aa, Artificial sequence
  Synthetic sequence
EVCSEQAETGPCRAGISRWYFDVTEGKCAPFFYGGCGGNRNNFDTEEYCMAVCGSAI

SEQ ID NO: 18 - protein, 57 aa, Artificial sequence
  Synthetic sequence
EVCSEQAETGPCRAMFSRWYFDVTEGKCAPFFYGGCGGNRNNFDTEEYCMAVCGSAI

SEQ ID NO: 19 - protein, 57 aa, Artificial sequence
  Synthetic sequence
EVCSEQAETGPCRAMFSRWYFDVTEGKCAPFVYGGCGGNRNNFDTEEYCMAVCGSAI

SEQ ID NO: 20 - protein, 57 aa, Artificial sequence
  Synthetic sequence
EVCSEQAETGPCRAGISRWYFDVTEGKCAPFVYGGCGGNRNNFDTEEYCMAVCGSAI

SEQ ID NO: 21 - protein, 57 aa, Artificial sequence
  Synthetic sequence
EVCSEQAETGPCRAGFSRWYFDVTEGKCAPFFYGGCGGNRNNFDTEEYCMAVCGSAI

SEQ ID NO: 22 - protein, 57 aa, Artificial sequence
  Synthetic sequence
EVCSEQAETGPCRAMISRWYFDVTEGKCAPFVYGGCGGNRNNFDTEEYCMAVCGSAI

SEQ ID NO: 23 - protein, 57 aa, Homo sapiens
EVCSEQAETGPCRAMISRWYFDVTEGKCAPFFYGGCGGNRNNFDTEEYCMAVCGSAI

SEQ ID NO: 24 - DNA, 60 bp, Artificial sequence
  Synthetic sequence
gatggtatttcgatgttactgaaggtaaatgtgctccattcttctatggtggttgtggtg

SEQ ID NO: 25 - DNA, 72 bp, Artificial sequence
  Synthetic sequence
ccacaaacagccatacaatattcttcagtatcgaaattatttctattaccaccacaaccaccatagaagaat

SEQ ID NO: 26 - DNA, 73 bp, Artificial sequence
  Synthetic sequence
gaagtttgttctgaacaagctgaaactggtccatgtagagctatgatttctagatggtatttcgatgttactg

SEQ ID NO: 27 - DNA, 64 bp, Artificial sequence
  Synthetic sequence
ggaaagccaatggtttatctggcaaggatccaatagcagaaccacaaacagccatacaatattc

SEQ ID NO: 28 - DNA, 66 bp, Artificial sequence
  Synthetic sequence
ggtggttctggtggtggtggttctggtggtggtggtctgctagcgaagtttgttctgaacaagctg

SEQ ID NO: 29 - DNA, 69 bp, Artificial sequence
  Synthetic sequence
gagctattacaagtcctcttcagaaataagcttttgttcagatggatcttggaaagccaatggtttatc

SEQ ID NO: 30 - DNA, 73 bp, Artificial sequence
  Synthetic sequence
gaagtttgttctgaacaagctgaaactggtccatgtagagctggtttttctagatggtatttcgatgttactg

SEQ ID NO: 31 - DNA, 73 bp, Artificial sequence
  Synthetic sequence
gaagtttgttctgaacaagctgaaactggtccatgtagagctggtatttctagatggtatttcgatgttactg

SEQ ID NO: 32 - DNA, 73 bp, Artificial sequence
  Synthetic sequence
gaagtttgttctgaacaagctgaaactggtccatgtagagctatgttttctagatggtatttcgatgttactg

SEQ ID NO: 33 - DNA, 73 bp, Artificial sequence
  Synthetic sequence
ccacaaacagccatacaatattcttcagtatcgaaattatttctattaccaccacaaccaccatagacgaatg

SEQ ID NO: 34 - DNA, 64 bp, Artificial sequence
  Synthetic sequence
agcgtatacgtagactataaggatgacgacgacaaagaattcgaagtttgttctgaacaagctg

SEQ ID NO: 35 - DNA, 61 bp, Artificial sequence
  Synthetic sequence
atagtttagcggccgcatgatggtggtgatggtgcctaggaatagcagaaccacaaacagc

SEQ ID NO: 36 - DNA, 34 bp, Artificial sequence
  Synthetic sequence
cggagcgaattcgaagtttgttctgaacaagctg

SEQ ID NO: 37 - DNA, 33 bp, Artificial sequence
  Synthetic sequence
cgctaccctaggaatagcagaaccacaaacagc

SEQ ID NO: 38 - DNA, 21 bp, Artificial sequence
  Synthetic sequence
gactggttccaattgacaagc

SEQ ID NO: 39 - DNA, 21 bp, Artificial sequence
  Synthetic sequence
gcaaatggcattctgacatcc

SEQ ID NO: 40 - protein, 57 aa, Artificial sequence; identical to SEQ ID NO: 1
  synthetic sequence
  MISC_FEATURE (9)..(9): X is cysteine, valine or threonine
  MISC_FEATURE (15)..(15): X is glycine or cysteine
  MISC_FEATURE (16)..(16): X is phenylalanine
  MISC_FEATURE (17)..(17): X is serine
  MISC_FEATURE (27)..(27): X is lysine or leucine
  MISC_FEATURE (32)..(32): X is is cysteine